(19) United States
(12) Patent Application Publication
(10) Pub. No.: US 2022/0361755 A1
(43) Pub. Date: Nov. 17, 2022

(54) SECURELY EXCHANGING INFORMATION BETWEEN A MEDICAL DEVICE AND A MOBILE COMPUTING DEVICE USING VISUAL INDICATORS

(71) Applicant: ZOLL Medical Corporation, Chelmsford, MA (US)

(72) Inventors: Wayne F. Stanley, Merrimack, NH (US); Guy R. Johnson, Wilton, NH (US); Binwei Weng, Andover, MA (US); Gary A. Freeman, Waltham, MA (US); Ulrich R. Herken, Medford, MA (US)

(21) Appl. No.: 17/743,806

(22) Filed: May 13, 2022

Related U.S. Application Data
(60) Provisional application No. 63/188,504, filed on May 14, 2021.

(51) Int. Cl. A61B 5/00; G16H 40/67

(52) U.S. Cl. CPC A61B 5/0017; A61B 5/7285; A61B 5/0006; G16H 40/67

(57) ABSTRACT

A medical system is provided. The medical system includes a medical device and a mobile computing device. The medical device includes at least one physiologic sensor configured to acquire physiological signals from a patient, at least one processor coupled to the at least one physiologic sensor, and at least one optical code encoded with encrypted data. The mobile computing device includes a camera and one or more processors coupled to the camera and configured to acquire one or more images of the at least one optical code, decode the one or more images of the at least one optical code to generate a copy of the encrypted data, decrypt the copy of the encrypted data to generate decrypted data, and process the decrypted data to establish an operable connection between the mobile computing device and the medical device.

### RELATED APPLICATION

This application claims priority under 35 U.S.C. § 119 (e) to U.S. Provisional Application Ser. No. 63/188,504, titled “SECURELY EXCHANGING INFORMATION BETWEEN A MEDICAL DEVICE AND A MOBILE COMPUTING DEVICE USING VISUAL INDICATORS,” filed May 14, 2021, which is hereby incorporated herein by reference in its entirety.

### BACKGROUND

Medical devices that can assist a health care provider in the administration of cardiopulmonary resuscitation (CPR) and related treatment include automated resuscitation systems and automated defibrillators, among others. Some treatment devices intelligently monitor a patient's condition, periodically provide instructions regarding treatment, store information related to treatment of the patient, and even administer treatment to the patient autonomously. For example, automated defibrillators include electrodes that can be coupled to the patient's skin to acquire electrical signals generated by a patient's cardiac activity. These electrical signals may be referred to as electrocardiogram (ECG) signals. Automated defibrillators also include circuitry that can analyze the acquired electrocardiogram signals in an attempt to categorize the analyzed signals into one of several predefined cardiac rhythms including, for example, a normal sinus rhythm or arrhythmias such as ventricular fibrillation (VF), ventricular tachycardia (VT), atrial fibrillation (AF), tachycardia, bradycardia, asystole, and pulseless electrical activity (PEA), to name a few. Exhibition of certain arrhythmias can indicate a life-threaten condition that can be treatable via electrotherapy. For example, a cardiac condition indicated by ventricular fibrillation can be treated by a defibrillating shock to the patient's myocardium. As such, some automated defibrillators include circuitry that can discharge electrotherapy automatically, or under control of a health care provider. These automated defibrillators include, or have access to, an energy source to power the electrotherapy and electrodes connected to the energy source through which the electrotherapy can be discharged. Occasionally, treatment of a patient using an automated defibrillator can be provided by a bystander prior to arrival of a health care provider.

To guide a health care provider in the administration of CPR, some automated defibrillators include a user interface to output instructions to the health care provider. These instructions can include prompts to begin a cycle of CPR, feedback to help pace compressions, prompts to administer rescue breaths, and prompts to pause CPR so that the automated defibrillator can analyze the patient's ECG signals to provide further instructions. These further instructions can include directions to administer subsequent cycles of CPR and/or to administer electrotherapy to the patient. Additionally, some medical treatment devices can be configured to provide historic treatment information to a health care provider related to treatment provided to a patient prior to the arrival of the health care provider.

### SUMMARY

In at least one example, a medical system is provided. The medical system includes a medical device and a mobile computing device. The medical device includes at least one physiologic sensor configured to acquire physiological signals from a patient, at least one processor coupled to the at least one physiologic sensor, and at least one optical code encoded with encrypted data. The mobile computing device includes a camera and one or more processors coupled to the camera and configured to acquire one or more images of the at least one optical code, decode the one or more images of the at least one optical code to generate a copy of the encrypted data, decrypt the copy of the encrypted data to generate decrypted data, and process the decrypted data to establish an operable connection between the mobile computing device and the medical device.

Implementations of the medical system can include one or more of the following features.

In examples of the medical system, the medical device can further include at least one network interface coupled to the at least one processor, the mobile computing device can include one or more network interfaces coupled to the one or more processors, the encrypted data can include network connection information, and to process the decrypted data can include to establish a connection between the mobile computing device and the medical device via the one or more network interfaces and the at least one network interface using the network connection information. In some examples, the network connection information can include one or more of security credentials, an identifier of the medical device, or an identifier of a network associated with the medical device. In some examples, the medical device can be further configured to exchange information with the mobile computing device, the exchanged information including at least one of device readiness information, caregiver performance data, physiological data, or event marker data.

In examples of the medical system, the medical device can include one or more of an automated external defibrillator, a defibrillator/monitor, a wearable defibrillator, a ventilator, a resuscitation system, a cardiac monitoring device, or a CPR monitoring device.

In examples of the medical system, the medical device can further include at least one display coupled to the at least one processor and the at least one processor can be configured to encrypt sensitive data to generate the encrypted data, encode the encrypted data and public data within the at least one optical code, and output, via the at least one display, the at least one optical code encoded with the encrypted data and the public data. In some examples, to decode the one or more images can include to decode the one or more images of the at least one optical code to generate the copy of the encrypted data and a copy of the public data, and the one or more processors are further configured to process the copy of the public data.

In examples of the medical system, the medical device can further include at least one display coupled to the at least one processor and the at least one processor can be configured to encrypt data to generate the encrypted data and output, via the at least one display, the at least one optical code encoded with the encrypted data. In some examples, the medical device can further include at least one network interface coupled to the at least one processor, the mobile computing device can include one or more network interfaces coupled to the one or more processors, the data can include network connection information, and to process the decrypted data can include to establish a connection between the mobile computing device and the medical device via the one or more network interfaces and the at least one network interface using the network connection information. In some examples, the mobile computing device can further include a user interface, the data can include encounter information, and to process the decrypted data can include to output the encounter information via the user interface. In some examples, the at least one physiologic sensor can include at least one ECG sensor configured to acquire transcutaneous ECG signals from the patient and the encounter information specifies one or more of initiation time and duration of an encounter documented by the encounter information, an arrhythmia condition of the patient detected during the encounter, treatment administered to the patient during the encounter, or efficacy of the treatment administered to the patient during the encounter. In some examples, the medical device can further include at least one therapy electrode configured to discharge transcutaneous electrotherapy to a myocardium of the patient and the encounter information further specifies one or more of a number of discharges administered to the patient during the encounter and whether the any of the one or more discharges resulted in conversion of the arrhythmia condition of the patient. In some examples, the medical device can further include at least one treatment sensor configured to monitor delivery of CPR to the patient and the encounter information specifies one or more of initiation time and duration of an encounter documented by the encounter information, emergency medical responder name information, CPR performance of the emergency medical responder, compression data and averages for a period of time, target treatment information, post shock pause values, pre-shock pause values, total length of treatment provided, or efficacy of treatment administered to the patient during the encounter. In some examples, the medical device can further include a ventilator including at least one flow sensor and at least one pressure sensor configured to measure air flow rates delivered to the patient and the encounter information specifies one or more of initiation time and duration of an encounter documented by the encounter information, breaths per minute as delivered to the patient and measured by the at least one flow sensor, air volume per breath as delivered to the patient and measured by the at least one flow sensor, air volume exhausted by the patient as measured by the at least one flow sensor, ventilator settings, or efficacy of treatment administered to the patient during the encounter.

In some examples of the medical system, the mobile computing device can further include a user interface, the data can include device readiness information, and to process the decrypted data can include to output the device readiness information via the user interface. In some examples, the device readiness information can specify one or more of a result of a self-test executed by the medical device, electrode expiration information, an amount of power remaining in a battery of the medical device, or status of network connectivity of the medical device. In some examples, to output the at least one optical code can include to encode the encrypted data within the at least one optical code. In some examples, the at least one optical code can include a plurality of optical codes.

In examples of the medical system, the mobile computing device can further include one or more light emitting devices coupled to the one or more processors, the medical device can further include at least one light sensor, the one or more processors can be further configured to encode new data as one or more modulations of the one or more light emitting devices and transmit the one or more modulations, and the at least one processor can be configured to acquire the one or more modulations via the at least one light sensor and demodulate the one or more modulations to generate a copy of the new data for processing.

In examples of the medical system, the medical device can further include at least one light emitting device positioned within the at least one optical code and the at least one processor can be configured to encrypt additional data to generate additional encrypted data, encode the additional encrypted data as a plurality of modulations of the at least one light emitting device, and transmit, via the at least one light emitting device, the plurality of modulations. In some examples, the medical device can further include at least one network interface coupled to the at least one processor, the mobile computing device can further include a user interface coupled to the one or more processors and one or more network interfaces coupled to the one or more processors, the data can include network connection information, the additional data can include encounter information, to process the decrypted data can include to establish a connection between the mobile computing device and the medical device via the one or more network interfaces and the at least one network interface using the network connection information, and the one or more processors can be further configured to demodulate the plurality of modulations to generate a copy of the additional encrypted data, decrypt the copy of the additional encrypted data to generate a copy of the additional data, and output, via the user interface, a copy of the encounter information from the copy of the additional data.

In examples of the medical system, the mobile computing device can further include one or more network interfaces coupled to the one or more processors and the one or more processors can be further configured to receive a request for the encrypted data from a trusted mobile computing device via the one or more network interfaces and transmit, in response to reception of the request, the encrypted data to the trusted mobile computing device via the one or more network interfaces. In some examples, the trusted mobile computing device is a wearable device.

In examples of the medical system, the mobile computing device can further include a user interface and the one or more processors can be further configured to receive a request for the at least one optical code from a trusted mobile computing device and display, in response to reception of the request, the at least one optical code via the user interface.

In another example, a medical device is provided. The medical device includes at least one display, at least one physiologic sensor configured to acquire physiological signals from a patient, and at least one processor coupled to the at least one display and the at least one physiologic sensor. The at least one processor is configured to identify data targeted for representation by at least one optical code, encrypt the data to generate encrypted data, encode the encrypted data within the at least one optical code, and output, via the at least one display, the at least one optical code.

Implementations of the medical device can include one or more of the following features.

In examples, the medical device can further include at least one network interface coupled to the at least one processor, wherein the data targeted for representation can include network connection information. In some examples, the network connection information can include one or more of security credentials, an identifier of the medical device, or an identifier of a network associated with the medical device.

In examples of the medical device, the at least one processor can be further configured to identify new data targeted for representation by at least one new optical code, the new data specifying new network connection information, encrypt the new data to generate new encrypted data, encode the new encrypted data within the at least one new optical code, and output, via the at least one display, the at least one new optical code.

In examples, the medical device can further include one or more of an automated external defibrillator, a defibrillator/monitor, a wearable defibrillator, a ventilator, a resuscitation system, a cardiac monitoring device, or a CPR monitoring device.

In examples of the medical device, the data targeted for representation can include sensitive data and the at least one processor can be further configured to encode public data within the at least one optical code. In some examples, the sensitive data can specify one or more of protected health information, demographic information regarding the patient, or information regarding a user of the medical device other than the patient.

In examples of the medical device, the data targeted for representation can include encounter information. In some examples, the at least one physiologic sensor can include at least one ECG sensor configured to acquire transcutaneous ECG signals from the patient and the encounter information can specify one or more of initiation time and duration of an encounter documented by the encounter information, an arrhythmia condition of the patient detected during the encounter, treatment administered to the patient during the encounter, or efficacy of the treatment administered to the patient during the encounter. In some examples, the medical device can further include at least one therapy electrode configured to discharge transcutaneous electrotherapy to a myocardium of the patient and the encounter information further specifies one or more of a number of discharges administered to the patient during the encounter and whether the any of the one or more discharges resulted in conversion of the arrhythmia condition of the patient. In some examples, the medical device can include at least one treatment sensor configured to monitor delivery of CPR to the patient and the encounter information can specify one or more of initiation time and duration of an encounter documented by the encounter information, emergency medical responder name information, CPR performance of the emergency medical responder, compression data and averages for a period of time, target treatment information, post shock pause values, pre-shock pause values, total length of treatment provided, or efficacy of treatment administered to the patient during the encounter. In some examples, the medical device can include at least one flow sensor configured to measure air flow rates delivered to the patient and the encounter information can specify one or more of initiation time and duration of an encounter documented by the encounter information, breaths per minute as delivered to the patient and measured by the at least one flow sensor, air volume per breath as delivered to the patient and measured by the at least one flow sensor, air volume exhausted by the patient as measured by the at least one flow sensor, or efficacy of treatment administered to the patient during the encounter.

In examples of the medical device, the data targeted for representation can include device readiness information. In some examples, the device readiness information can specify one or more of a result of a self-text executed by the medical device, electrode expiration information, an amount of power remaining in a battery of the medical device, or status of network connectivity of the medical device.

In examples of the medical device, the at least one optical code can include a plurality of optical codes. In some examples, to output the at least one optical code can include to output the plurality of optical codes in a sequence.

In examples, the medical device can include at least one light sensor, wherein the at least one processor can be further configured to acquire one or more modulations of one or more light emitting devices via the at least one light sensor and demodulate the one or more modulations to generate new data for processing.

In another example, a second medical device is provided. The additional medical device includes at least one display, at least one physiologic sensor configured to acquire physiological signals from a patient, and at least one processor coupled to the at least one display and the at least one physiologic sensor and configured to output, via the at least one display, a plurality of optical codes in a sequence, at least one optical code of the plurality of optical codes being encoded with encrypted data.

Implementations of the second medical device can include one or more of the follow features.

In examples, the second medical device can include at least one network interface coupled to the at least one processor, wherein the encrypted data can include network connection information. In some examples, the network connection information can include one or more of security credentials, an identifier of the medical device, or an identifier of a network associated with the medical device. In some examples, the at least one processor can be further configured to identify new data targeted for rendering within at least one new optical code, the new data specifying new network connection information, encrypt the new data to generate new encrypted data, encode the new encrypted data within the at least one new optical code, and render, via the at least one display, the at least one new optical code within the plurality of optical codes.

In examples, the second medical device can include one or more of an automated external defibrillator, a defibrillator/monitor, a wearable defibrillator, a ventilator, a resuscitation system, a cardiac monitoring device, or a CPR monitoring device.

In examples of the second medical device, the encrypted data can include sensitive data and the at least one processor can be further configured to encode public data within the at least one optical code. In some examples, the sensitive data can specify one or more of protected health information, demographic information regarding the patient, or information regarding a user of the medical device other than the patient.

In examples of the second medical device, the encrypted data can include encounter information. In some examples, the at least one physiologic sensor can include at least one ECG sensor configured to acquire transcutaneous ECG signals from the patient and the encounter information specifies one or more of initiation time and duration of an encounter documented by the encounter information, an arrhythmia condition of the patient detected during the encounter, treatment administered to the patient during the encounter, or efficacy of the treatment administered to the patient during the encounter. In some examples, the second medical device can include at least one therapy electrode configured to discharge transcutaneous electrotherapy to a myocardium of the patient and the encounter information can further specify one or more of a number of discharges administered to the patient during the encounter and whether the any of the one or more discharges resulted in conversion of the arrhythmia condition of the patient. In some examples, the second medical device can further include at least one treatment sensor configured to monitor delivery of CPR to the patient and the encounter information can specify one or more of initiation time and duration of an encounter documented by the encounter information, emergency medical responder name information, CPR performance of the emergency medical responder, compression data and averages for a period of time, target treatment information, post shock pause values, pre-shock pause values, total length of treatment provided, or efficacy of treatment administered to the patient during the encounter. In some examples, the second medical device can include at least one flow sensor configured to measure air flow rates delivered to the patient and the encounter information specifies one or more of initiation time and duration of an encounter documented by the encounter information, breaths per minute as delivered to the patient and measured by the at least one flow sensor, air volume per breath as delivered to the patient and measured by the at least one flow sensor, air volume exhausted by the patient as measured by the at least one flow sensor, or efficacy of treatment administered to the patient during the encounter.

In examples of the second medical device, the encrypted data can include device readiness information. In some examples, the device readiness information can specify one or more of a result of a self-text executed by the medical device, an amount of power remaining in a battery of the medical device, or status of network connectivity of the medical device.

In examples, the second medical device can include at least one light sensor, wherein the at least one processor can be further configured to acquire one or more modulations of one or more light emitting devices via the at least one light sensor and demodulate the one or more modulations to generate new data for processing.

Still other aspects, examples, and advantages of these aspects and examples, are discussed in detail below. Moreover, it is to be understood that both the foregoing information and the following detailed description are merely illustrative examples of various aspects and features and are intended to provide an overview or framework for understanding the nature and character of the claimed aspects and examples. Any example or feature disclosed herein can be combined with any other example or feature. References to different examples are not necessarily mutually exclusive and are intended to indicate that a particular feature, structure, or characteristic described in connection with the example can be included in at least one example. Thus, terms like “other” and “another” when referring to the examples described herein are not intended to communicate any sort of exclusivity or grouping of features but rather are included to promote readability.

### BRIEF DESCRIPTION OF THE DRAWINGS

Various aspects of the disclosure are discussed below with reference to the accompanying figures, which are not intended to be drawn to scale. The figures are included to provide an illustration and a further understanding of various examples and are incorporated in and constitute a part of this specification but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure. A quantity of each component in a particular figure is an example only and other quantities of each, or any, component could be used.

FIG. 1 illustrates a sample system for exchanging information between a medical device and a mobile computing device using a visual indicator in accordance with at least one example disclosed herein.

FIG. 2A illustrates a sample medical device configured to display a visual indicator in accordance with at least one example disclosed herein.

FIG. 2B illustrates a second sample medical device configured to display a visual indicator in accordance with at least one example disclosed herein.

FIG. 3 illustrates a sample system for encoding encrypted data into a visual indicator in accordance with at least one example disclosed herein.

FIG. 4 illustrates a sample system for encoding encrypted data and public data into a visual indicator in accordance with at least one example disclosed herein.

FIG. 5 illustrates a sample system for encoding data into a visual indicator including both a static portion and a dynamic portion in accordance with at least one example disclosed herein.

FIG. 6 illustrates a sample visual indicator including both a static portion and a dynamic portion in accordance with at least one example disclosed herein.

FIG. 7 illustrates a sample system-based process flow for exchanging encrypted information between a medical device and a mobile computing device in accordance with at least one example disclosed herein.

FIG. 8A illustrates a sample system-based process flow for connecting multiple mobile computing devices to a medical device using a gateway device in accordance with at least one example disclosed herein.

FIG. 8B illustrates a sample system-based process flow for connecting a wearable computing device to a mobile computing device to access information collected by a medical device in accordance with at least one example disclosed herein.

FIGS. 8C and 8D illustrate sample screenshots depicting information displayed on a remote computing device in accordance with at least one example disclosed herein.

FIG. 9 illustrates a sample system-based process flow including a health care provider operably connecting their mobile computing device to a medical device in accordance with at least one example disclosed herein.

FIG. 10A illustrates a system-based process flow depicting transportation of a patient to a hospital and establishing communications between a medical device providing treatment to the patient and one or more mobile computing devices in accordance with at least one example disclosed herein.

FIG. 10B illustrates a system-based process flow depicting transportation of a patient to a hospital and establishing communications between a second medical device providing treatment to the patient and one or more mobile computing devices in accordance with at least one example disclosed herein.

FIG. 11 illustrates a flow diagram depicting a process for securely transmitting information between a medical device and a mobile computing device in accordance with at least one example disclosed herein.

FIG. 12 illustrates a flow diagram depicting a process for generating a visual indicator by a medical device in accordance with at least one example disclosed herein.

FIG. 13A illustrates a flow diagram depicting a process for generating a visual indicator including both public and sensitive data in accordance with at least one example disclosed herein.

FIG. 13B illustrates a flow diagram depicting a process for a mobile computing device to decode a visual indicator and accessing public information in accordance with at least one example disclosed herein.

FIGS. 14A and 14B illustrate flow diagrams depicting processes for exchanging information between a medical device and a mobile computing device in accordance with at least one example disclosed herein.

FIG. 15 illustrates a process for a mobile computing device to function as a gateway device in accordance with at least one example disclosed herein.

FIG. 16 illustrates a process for establishing a secure connection between a medical device in a mobile computing device in accordance with at least one example disclosed herein.

FIG. 17 illustrates a process for transmitting high priority data between a medical device and a mobile computing device in accordance with at least one example disclosed herein.

FIG. 18A illustrates a sample quick response (QR) code in accordance with at least one example disclosed herein.

FIG. 18B illustrates a sample QR code sequence in accordance with at least one example disclosed herein.

FIG. 19 is a schematic block diagram of examples of computing and medical device components in accordance with at least one example disclosed herein.

FIG. 20 is a schematic block diagram of a medical treatment device in accordance with at least one example disclosed herein.

FIGS. 21A-21E are illustrative data structures showing data stored by a medical device in accordance with at least one example disclosed herein.

### DETAILED DESCRIPTION

As summarized above, certain examples described herein are directed to medical devices that are configured to securely transfer information to a mobile computing device using at least one optical code. This optical code can be used to establish a communication channel between a medical device and the mobile computing device. For example, the transferred information can be used by the mobile computing device to establish a secure connection to the medical device such that information collected by the medical device related to monitoring and or treatment of a patient can be transferred to the mobile computing device for analysis by a user of the mobile computing device. Other information can also be provided, such as device readiness information, so that a user can determine a current status of the medical device.

In certain situations, treatment of a patient using a medical device may begin before a more experienced health care provider is on the scene and ready to treat the patient. For example, a patient experiencing a cardiac arrhythmia may be treated by a bystander using an automated external defibrillator (AED) The AED may include instructions for positioning one or more therapy electrodes on the patient and providing one or more therapy shocks to the patient in response to detection of the cardiac arrhythmia. While the AED can collect information related to monitoring and treatment of the patient, this information may be difficult to securely transfer to a mobile computing device associated with the health care provider once the health care provider arrives on scene. Similarly, while the bystander can attempt to explain or provide verbal information related to the treatment of the patient, the health care provider may receive greater benefit from obtaining the information directly from the medical device itself.

The scenario articulated above illustrates one example where secure communication between a medical device and a mobile computing device associated with the health care provider is advantageous to the continued treatment of the patient. However, if the medical device and the mobile computing device do not have previous knowledge of each other and their network connectivity information, establishing a quick and secure connection between the two can be difficult and/or time consuming. The systems and processes as described herein are directed to providing quick, dynamic, and secure information from a medical device to a mobile computing device that can be used by the mobile computing device to establish a secure communication link with the medical device, and to securely exchange information such as patient treatment information with the medical device.

In some examples as described herein, the medical device can be configured to generate a dynamic optical code, such as a QR code, wherein the optical code can be dynamic or static in nature, that includes encrypted information relating to establishing a connection with the medical device. A mobile computing device can be configured to capture an image of the optical code, decode the optical code to determine the encrypted information, and decrypt the encrypted information using a decryption key accessible by, for example, an application running on the mobile computing device. In certain implementations the application can be provided by or otherwise distributed by the manufacturer of the medical device so that health care providers qualified and trained to use the medical device have access to the application and encrypted information contained in the optical codes as described herein.

For example, the application running on the mobile computing device can decrypt the encrypted information to determine network connectivity information such as network ID, device ID, session information, security information, and other similar connection information associated with the medical device that the medical device has included in the encrypted information in the optical code. The mobile computing device can access this network connection information to establish direct or indirect connection with the medical device as described herein, thereby facilitating exchange of information between the mobile computing device and the medical device.

In certain examples, the encrypted information can include additional information beyond network connection information such as patient-specific or medical event information related to the monitoring and/or treatment of the patient by the medical device. To continue the above example, when the device is an AED, the patient-specific information can include information about usage of the AED to treat the patient, whether electrodes have been applied to the patient (e.g., via recording of patient impedance through the electrodes), any cardiac arrhythmia experienced by the patient and detected by the medical device, any therapeutic shocks delivered to the patient by the medical device, timing information related to the therapy shocks delivered to the patient, energy information related to the therapy shocks delivered to the patient, chest compression information (e.g., information indicating whether chest compressions have been given to the patient, chest compression performance metrics, such as depth and rate of compressions, etc.), and other similar patient-specific information regarding monitoring and/or treatment of the patient by the medical device.

In some examples, a mobile computing device can function as a gateway device to facilitate communication between a medical device and a secondary mobile computing device such as a tablet computing device, a laptop computing device, a wearable computing device such as a smart watch, and other similar mobile computing devices. In such an example, the gateway device establishes a network connection with the medical device as described herein. In response to a request from a trusted device to establish a network connection with the medical device, the gateway device can provide the requesting trusted device with information that the trusted device can use to establish a connection with the medical device. For example, a primary mobile computing device can use information obtained from one or more optical codes displayed on the medical device to establish a secure communication channel with the medical device. After a period of time, one or more other secondary mobile computing devices (e.g., tablet, watch, phone, laptop, other computer) can establish a communication channel with the primary mobile computing device to pull relevant information originating from the medical device. Alternatively, the one or more other secondary mobile computing devices can use the connection information from the primary mobile computing device to establish a direct communication channel with the medical device to pull the relevant information.

In certain examples as described herein, a medical device can be configured to output or otherwise provide information using one or more optical codes such as the QR code described above. In certain implementations, the optical code can include a combination of publicly-accessible information such as timing information, location information, device name information, and other similar publicly-accessible information in combination with secure information encrypted by the processor of the medical device as noted above and described herein, such as medical event information and device status information. In such an example, the optical code can include both the publicly-accessible information and the secure information, encoded into a single optical code and/or multiple optical codes. In some examples, the optical code(s) can include both the static component as well as a dynamic component. For example, the static component can include a non-changing portion of the optical code(s) that includes a portion of the publicly-accessible information, and the dynamic component that includes a changing portion of the optical code(s) that includes, for example, the secure information.

The processes and apparatus disclosed herein for encoding and transmitting information using visual indicates can be implemented via a variety of medical devices. These medical devices may include, for example, external defibrillators/monitors, AEDs, ventilators, wearable defibrillators, and automated resuscitation systems. Examples of external defibrillators/monitors include the R SERIES® or X SERIES® defibrillators commercially available from ZOLL Medical Corporation of Chelmsford, Mass. External defibrillators/monitors are often used by hospital personnel, emergency medical services, and the military. Examples of AEDs include the AED PLUS® automated external defibrillator or the AED PRO® automated external defibrillator commercially available from ZOLL Medical Corporation. AEDs are frequently seen in airports, public gymnasiums, schools, shopping areas and other public spaces. Examples of ventilators include the Z VENT® ventilators commercially available from ZOLL Medical Corporation of Chelmsford, Mass. Such ventilators are often used by emergency medical services, hospital personnel, and military. Examples of wearable defibrillators include the ZOLL® LIFEVEST® wearable cardioverter defibrillator commercially available from ZOLL Medical Corporation. Examples of automated resuscitation systems include the AutoPulse® chest compression device commercially available from the ZOLL Medical Corporation.

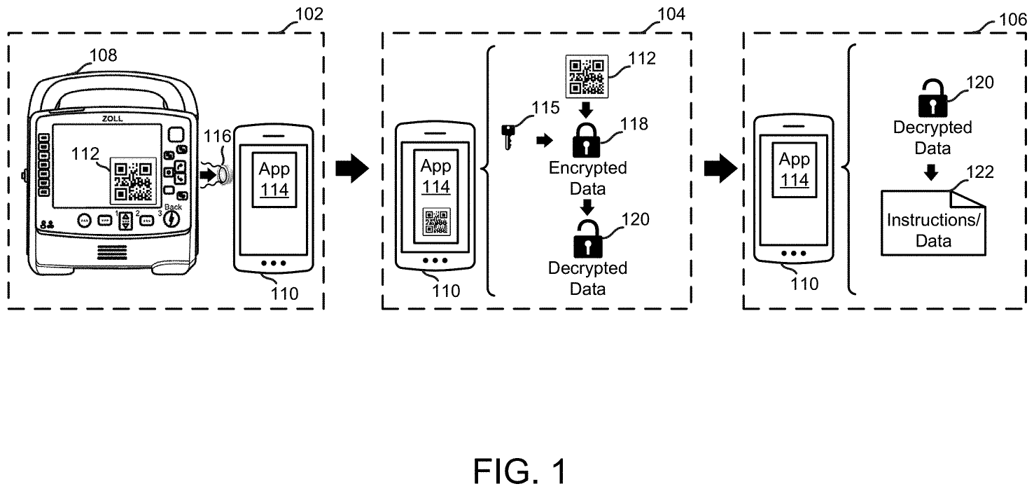

As described herein, the systems and methods can facilitate connection between at least one medical device and at least one additional device such as a personal computing device or other similar processing device. As shown in FIG. 1, this connection can include multiple states such as a state 102, a state 104, and a state 106. As shown in the state 102, a medical device 108 can be configured to automatically establish a connection with a mobile computing device 110 when the mobile computing device 110 scans an optical code such as a QR code 112 and processes the information embedded within the QR code 112. For example, the medical device 108 can be configured to display a visible marker or optical code such as the QR code 112. As further shown in FIG. 1, the mobile computing device 110 can include a camera or other similar image capture device 116 configured to capture an image of the QR code 112 as displayed on the medical device 108. Upon scanning the QR code 112, the mobile computing device 110 can establish a communicative connection with the medical device 108 and can also obtain other information relevant to the medical device 108, as discussed further herein.

It should be noted that the optical code as described herein is shown in the figures and addressed in the accompanying description as a QR code by way of example only. In implementations, additional and/or alternate optical codes can be used. For example, an optical code can include a one-dimensional code such as a bar code, a two-dimensional code such as a QR code, and other similar optical codes. The optical codes can be used in combination with other coding modalities such as color-based or time-divisional coding to enhance bandwidth, capacity, or error rates.

As further shown in FIG. 1, the QR code 112 can be generated to include information for processing by the mobile computing device 110 for establishing a direct communication link between the medical device 108 and the mobile computing device 110. More specifically, as described herein, the QR code 112 can include both encrypted and decrypted information used to establish a connection between the medical device 108 and the mobile computing device 110. The mobile computing device 110 can include a device-specific application 114 that is configured to receive a copy of the captured image of the QR code 112 from the image capture device 116 and process the image of the QR code 112 to decode the information contained within the QR code 112. For example, as described herein, the QR code 112 can include identification information related to the medical device 108, network connectivity information related to the medical device 108, patient-specific information related to a patient being treated by and/or using the medical device 108, as well as other information as described herein.

In order to properly decode the information contained within the QR code 112, the application 114 may include a decryption key or other similar cryptographic tool for use in decrypting the information contained within the QR code 112. For example, as shown in the state 104, the application 114 can be configured to decode the information contained within the QR code 112 to determine one or more pieces of encrypted data 118. Using a decryption key 115 associated with the application 114, the application 114 can decrypt the encrypted data 118 to generate decrypted data 120. For example, as described herein, the decrypted data 120 can include patient-specific data, connectivity data related to a network ID and/or other similar network information associated with the medical device 108, operational information related to the medical device 108, treatment information related to any treatment provided by and/or using the medical device 108 to the patient, and other similar sensitive data that is encrypted in order to protect the identity of a patient, treatment information related to the patient, and/or operational information related to the medical device 108.

More specifically, the encrypted data 118 can include device readiness information such as self-test information and results, electrode expiration information, remaining battery life, and other similar device readiness information. The encrypted data 118 can also include caregiver identification and performance data such as compression quality when administering CPR, ventilation quality when manually ventilating a patient, and other similar caregiver performance information. The encrypted data 118 can also include physiological data for a patient, which may be pertinent for a user to urgently receive before establishing a connection between the medical device 108 and the mobile computing device 110 or to receive while the connection is being established. Such pertinent data can include, for example, ECG information (e.g., a snapshot of an ECG waveform prior to defibrillation), blood oxygen information (e.g., SpO2 trending data), capnography measurement information (e.g., EtCO2 trending data), blood pressure data (e.g., non-invasive blood pressure (NIBP) trending data), and other similar physiological data. Additionally, the encrypted data 118 can include event marker information such as when did a specific treatment begin, what heart rhythm was detected prior to and after treatment, were any therapy shocks delivered to the patient, were any medications delivered to the patient, efficacy of treatment provided to the patient, and other similar event marker information.

In some examples, the medical device 108 can include a defibrillator. In such an example, the encrypted data 118 can include device-specific encounter information such as a number of shocks administered to the patient during the encounter, energy levels for any shocks discharged to the patient, whether the any of the one or more shocks resulted in conversion of the arrhythmia condition of the patient, and other similar encounter information. In some examples, the medical device 108 can include a treatment sensor configured to monitor delivery of CPR to the patient. In such an example, the encrypted data 118 can include device-specific encounter information such as initiation time and duration of an encounter documented by the encounter information, emergency medical responder name information, CPR performance of the emergency medical responder, compression data and averages for a period of time, target treatment information, post shock pause values, pre-shock pause values, total length of treatment provided, efficacy of treatment administered to the patient during the encounter, and other similar encounter information. In some examples, the medical device 108 can include a ventilator including at least one flow sensor and one pressure sensor configured to measure air flow rates delivered to the patient. In such an example, the encrypted data 118 can include device-specific encounter information such as initiation time and duration of an encounter documented by the encounter information, breaths per minute as delivered to the patient and measured by the at least one flow sensor, air volume per breath as delivered to the patient and measured by the at least one flow sensor, air volume exhausted by the patient as measured by the at least one flow sensor, efficacy of treatment administered to the patient during the encounter, ventilator settings (including, for example, mode, tidal volume, minute volume, breath rate, fraction of inspired oxygen, inspiratory/expiratory ratio, positive end expiratory pressure, and peak inspiratory pressure), and other similar encounter information.

In some examples, the encrypted data 118 can include patient-specific information such as patient demographic information, medical history, current medication information, height, weight, gender, and other patient-specific information.

Referring back to FIG. 1, as further shown in the state 106, the application 114 can analyze the now decrypted data 120 to determine one or more instructions and/or data 122 contained within the decrypted data 120. For example, the instructions and/or data 122 can include information related to establishing a direct connection between the medical device 108 and the mobile computing device 110, patient-specific information related to a patient being treated by the medical device 108, responder information related to a person using the medical device 108, operational information related to the medical device 108 such as network connectivity information, and/or other similar information that may be encrypted by the medical device 108 when generating the QR code 112.

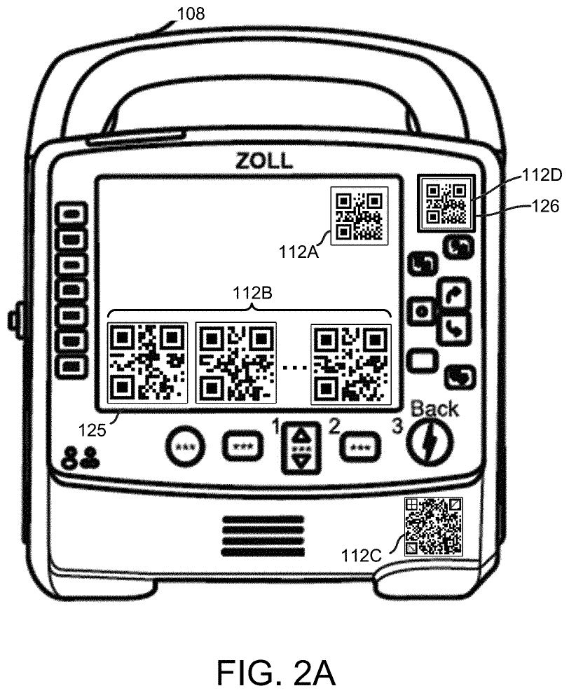

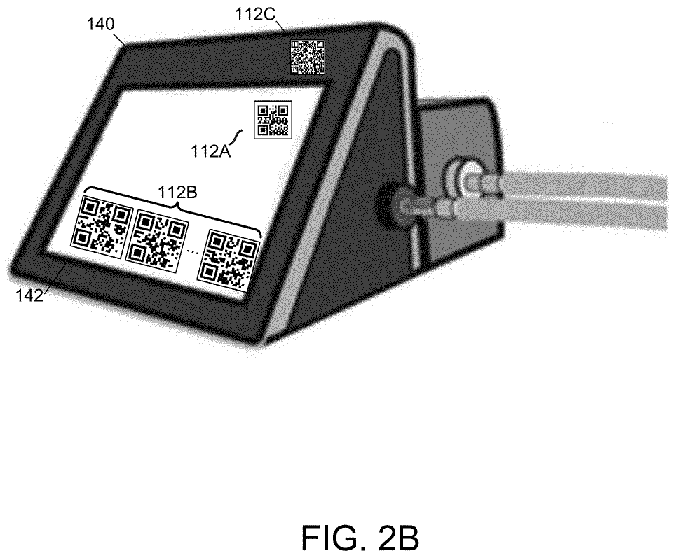

It should be noted that the embodiment as illustrated in FIG. 1 is provided by way of example only. For example, the position of the QR code 112 on the medical device 108 is shown as being displayed upon a screen of the medical device 108 as an example. In certain implementations, the QR code 112 can be displayed in various positions on the medical device 108 depending upon the size, shape, functionality, and other similar attributes of the medical device 108. For example as shown in FIG. 2A, a QR code can be displayed in various positions on the medical device 108, both as a dynamically updated QR code as well as a static QR code. As shown in FIG. 2A, one or more dynamically updated QR codes 112A and/or 112B can be displayed on an output interface such as a screen 125 of the medical device 108. Alternatively or additionally, the QR codes 112A and/or 112B can be displayed on a dedicated display location on the medical device separate from the main display. As described hereinbelow, the dynamically updated QR codes 112A and/or 112B can be generated by a processor of the medical device 108 to provide updated and/or patient-specific information to, for example, a medical responder and/or other technician using the medical device 108. In certain implementations, the QR code can be displayed as a series of QR codes 112B including alternate QR codes displayed simultaneously and/or a string of alternating QR codes shown overlapping in the same position on screen 125 of medical device 108. For example, each QR code as displayed in the series of QR codes 112B can be displayed on the screen 125 for one or more seconds before the screen 125 transitions to the next QR code in this series 112B. In certain implementations, within a series of QR codes such as series 112B, a portion of each QR code in the series 112B can remain static and display information that has not changed over time such as device information and network connectivity information. Similarly, a portion of each QR code in the series 112B can by dynamically updated to include information that is changing over time such as patient information, treatment information, device operation information, and other similar dynamic information as described herein.

In an example including a series of QR codes such as series 112B, the application (e.g., the application 114) on the user's mobile computing device (e.g., the mobile computing device 110) can be configured to continually scan the screen 125 of the medical device 108 to capture an image of each of the QR codes in the series 112B. Depending upon the image capture capability of the user's mobile computing device, and the display capabilities of the screen 125 of the medical device 108, the display time of each QR code in sequence 112B can be varied accordingly. For example, each QR code in series 112B can be displayed for about 0.10 seconds, 0.50 seconds, 1.0 seconds, 1.5 seconds, 2.0 seconds, 2.5 seconds, and other similar time periods. In some examples, the screen 125 can have a refresh rate of about 24 hz. In such an example, the screen 125 is configured to refresh 24 times per second. In this example, each of the QR codes in the series 112B can be displayed for one or more refresh periods. As such, a single QR code in the series 112B can be displayed for 1/24 of a second.

In some additional examples, a QR code can be positioned on the medical device 108 as a static QR code. For example, a QR code 112C can be displayed on the medical device 108 as a static QR code. The static QR code 112C can be applied, for example, to the housing of medical device 108 as a label, a sticker, a decal, and/or other similar permanent or semi-permanent applications such that the QR code 112C remains on the medical device 108 for a period of time until either updated and/or removed.

It should be noted that the position of the QR codes as shown in FIG. 2A is provided by way of example only. In certain implementations, the position of each individual QR code can be updated based upon the design, shape, size, and other attributes of the medical device 108. For example, the static QR code 112C can be positioned at various locations about the housing of the medical device 108. In certain implementations, an additional piece of the housing of the medical device 108 can be configured to cover the QR code 112C to prevent unauthorized access or manipulation of and/or tampering with the QR code 112C over time.

In some examples, the medical device 108 can include a secondary screen configured to solely display a QR code. For example, the QR code 112D can be displayed on a small, dedicated display 126 within the housing of the medical device 108. Such an arrangement provides for the medical device 108 to update the QR code 112D to provide a dynamic QR code while maintaining or reducing the amount of space of the screen 125 that would otherwise be used to display a QR code. For example, the dedicated display 126 can be a low power display such as an e-Ink display, a liquid crystal display (LCD), or other similar displays. Including the dedicated display 126 separate from the main display (e.g., screen 125) can be advantageous in that it can be separately controlled from the main display (and hence, can be continuously updated), and further can be configured to urgently provide key information for a user to immediately access without need to first establish a communication connection.

It should also be noted that medical device 108 is depicted in FIG. 2A by way of example only. In certain implementations, a medical device configured to display or otherwise output an optical code as described herein can include various other medical devices including, for example, a ventilator, a resuscitation system, a cardiac monitoring device, and/or a CPR monitoring device. For example, FIG. 2B illustrates a second medical device 140 that is configured to include one or more optical codes as described herein. In the example as illustrates in FIG. 2B, the second medical device 140 is shown as a ventilator by way of example only. As shown in FIG. 2B, and similar to FIG. 2A, one or more dynamically updated QR codes 112A and/or 112B can be displayed on an output interface such as a screen 142 of the medical device 140. As described hereinbelow, the dynamically updated QR codes 112A and/or 112B can be generated by a processor of the second medical device 140 to provide updated and/or patient-specific information to, for example, a responder and/or other technician using the second medical device 140. In certain implementations, the QR code can be displayed as a series of QR codes 112B including alternate QR codes displayed simultaneously or a string of QR codes shown overlapping in the same position on screen 142 of second medical device 140. In some additional examples, a QR code can be positioned on the second medical device 140 as a static QR code. For example, a QR code 112C can be displayed on the second medical device 140 as a static QR code. The static QR code 112C can be applied, for example, to the housing of the second medical device 140 as a label, a sticker, a decal, and/or other similar permanent or semi-permanent applications such that the QR code 112C remains on the second medical device 140 for a period of time until either updated or removed.

Optical codes, including the QR codes as described herein, can be used in combination with other encoding modalities such as color-based coding whereby one or more “pixels” of the QR code is further encoded with a distinct color, wavelength band or combination of wavelengths. For instance, if there are ten color values in the encoding, the channel capacity can be increased by that same amount, e.g., a 10-fold enhancement. In some examples, the color codes can be mapped to a camera photodetector color space such that the particular colors chosen are optimized for signal to noise ratio, e.g. equidistant within the color-space.

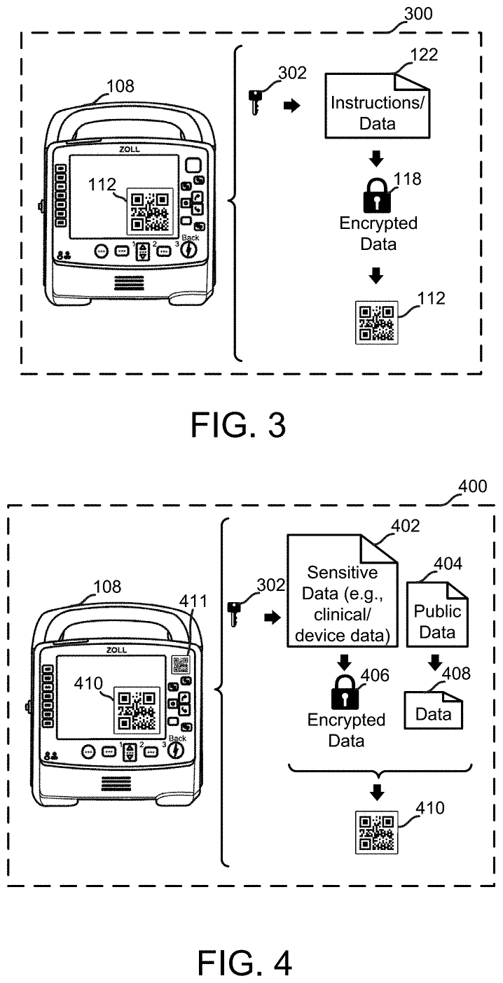

As described herein, and briefly mentioned above, a processor of a medical device such the medical device 108 can be configured to generate a dynamic QR code for display on the medical device 108. As shown in FIG. 3, in a sample system 300, the medical device 108 can be configured to generate and/or update the QR code 112 to include updated information during operation of the medical device 108. For example, the medical device 108 can include an encryption key 302 configured to encrypt at least a portion of the data to be displayed in the QR code 112. The processor of the medical device 108 can determine a set of instructions and/or data 122 that is to be encrypted for display within the QR code 112. The processor can encrypt the instructions/data 122 using the encryption key 302 to generate an encrypted data 118. The processor can further encode the encrypted data 118 into a dynamic QR code 112 for output on the display of the medical device 108 as described herein.

In an alternative embodiment, the processor of the medical device 108 can be configured to combine both encrypted data and publicly-accessible data into a dynamic QR code. For example, as shown in FIG. 4, in a sample system 400, the processor of the medical device 108 can be configured to generate a dynamic QR code 410 that includes both encrypted data and publicly-accessible data. As further shown in FIG. 4, the medical device can include an encryption key 302 similar to the encryption key 302 as described above in regard to FIG. 3. The processor of the medical device 108 can be configured to identify a portion of data 402 that is sensitive including, for example, clinical data related to the treatment and/or monitoring of a patient, operational information related to the operation of the medical device 108, connectivity information related to network ID or network access information for the medical device 108, and/or other similar sensitive data. The processor of the medical device 108 can also be configured to identify information 404 that is accessible to the public such as timing information, device information such as serial number and model, and other similar publicly-accessible data.

As further shown in FIG. 4, the processor of the medical device 108 can be configured to encrypt the sensitive data 402 using the encryption key 302 to generate an encrypted data 406. The processor of the medical device 108 can also process the public data information 404 to generate a set of publicly-accessible data 408 to be included in the dynamic QR code 410. The processor of the medical device 108 can combine the encrypted data 406 and the publicly-accessible data 408 to generate a temporary set of data and encode the temporary set of data into the dynamic QR code 410. As further shown in FIG. 4, the medical device 108 can be configured to display the dynamically generated QR code 410 on a main display screen of the medical device 108. In certain implementations, as described above in the discussion of FIG. 2A, a QR code 411 can be generated by the processor of the medical device 108 and displayed on a dedicated screen (e.g., screen 126 as shown in FIG. 2A and described above). For example, the processor of the medical device 108 can combine the encrypted data 406 and the publicly-accessible data 408 to generate a temporary set of data and encode the temporary set of data into the dynamic QR code 411 for display on the dedicated screen as described herein.

It should be noted that, using the processes as described herein, the processor of the medical device 108 can continually update the dynamically generated QR codes 410 and 411 and display an updated QR code such that, upon accessing the QR code, a responder and/or technician using the medical device 108 is presented with updated data.

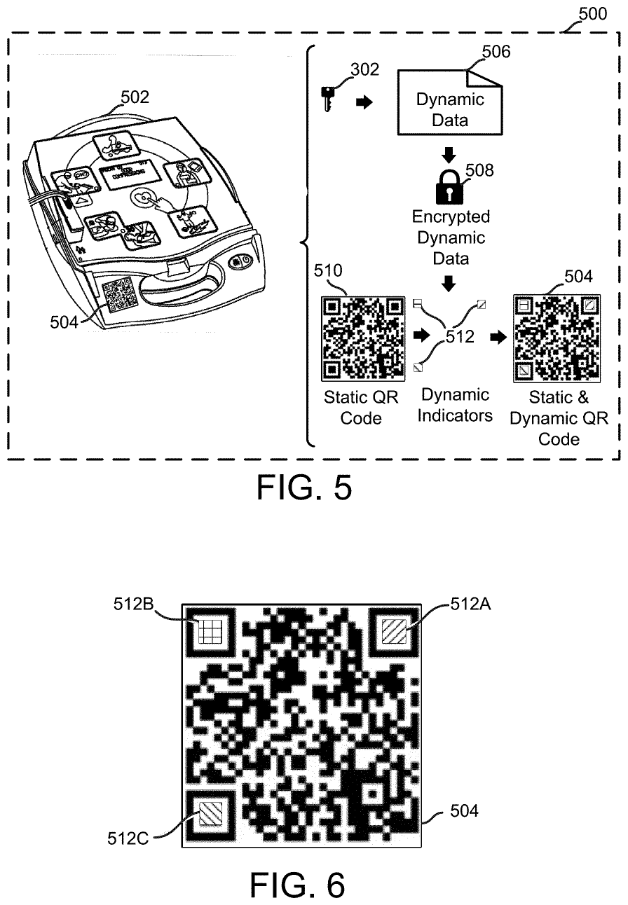

In certain implementations, a medical device may have limited display capabilities. For example, the medical device may be designed/configured such that it does not include a display configured to output a dynamically updated QR code. In such an example, the housing and/or output capabilities of the medical device can be updated such that at least a portion of the QR code provides a dynamic indication of updated information. For example as shown in FIG. 5, a medical device 502 can be configured to display a QR code 504 that includes both static data as well as dynamically updated data. In such an example, the QR code 504 can include a label, sticker, and/or other similarly adhered portion that includes the static, or unchanging, data to be included in the QR code. Additionally the housing about the static portion of the QR code 504 can be updated to include, for example, LED lights and/or other similar output devices configured to display dynamic information through for example displaying a blinking light pattern, alternating color light pattern, different lighting shades or colors, and other similar dynamic indications. As further shown in system 500 of FIG. 5, the processor of the medical device 502 can identify a set of dynamic data 506 to include in the dynamic portion of the QR code 504. Using the encryption key 302, the processor of the medical device 502 can encrypt the dynamic data 506 to generate an encrypted dynamic data 508. The processor of the medical device 502 can generate or otherwise determine one or more dynamic indicators 512 to be included in the QR code 504. The processor of the medical device 502 can cause the dynamic indicators 512 to be displayed along with the static QR code portion 510 such that the QR code 504 includes both the static QR code portion 510 and the dynamic indicators 512.

FIG. 6 illustrates a more detailed view of the QR code 504 including, for example, both the static QR code portion 510 as well as the dynamic indicators 512. More specifically, as shown in FIG. 6, the QR code 504 can include three specific dynamic indicators 512A, 512B, and 512C. However, it should be noted that three dynamic indicators are provided by way of example only. In certain implementations, each of the dynamic indicators 512 can be associated with, for example, a single LED or other similar output device configured to display or otherwise output dynamic information associated with the dynamic portion of the QR code 504. For example, each LED can be configured to display a single color light, alternate between colors of light, modulate or otherwise output a specific pattern of light, or otherwise output a dynamic portion of the QR code 504. When scanning the QR code 504, an image capture device can be configured to capture both the static portion 510 of the QR code 504 as well as the output of each of the dynamic indicators 512. As such, a single QR code 504 can be configured to display both static information as well as dynamic information. Using such a QR code, a medical responder and/or technician associated with or otherwise working with the medical device 502 can access the static information included in the QR code 504 as well as updated information related to, for example, operation of the medical device 502 that is included in the dynamic information of the QR codes 504 and provided by the dynamic indicators 512.

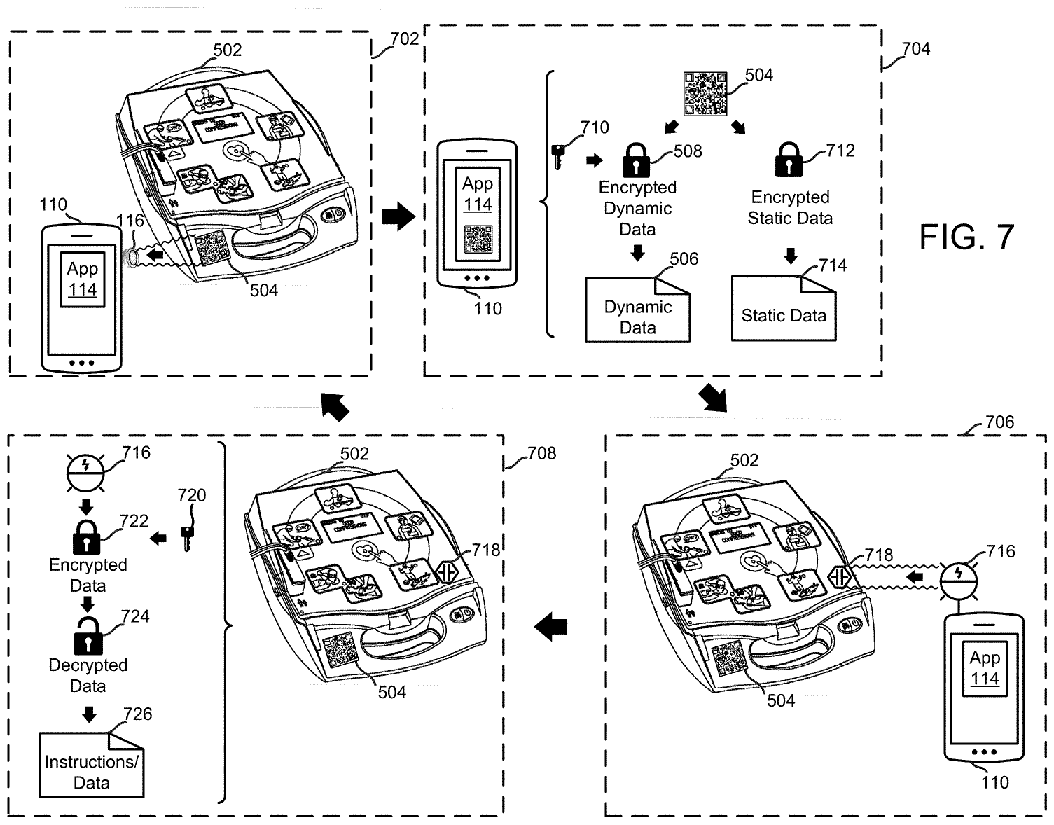

In addition to exchanging patient and operational information between a medical device and a user device, the processes as described herein can also be used to establish a direct connection between the user device and the medical device. For example, FIG. 7 shows a system-based process flow associated with establishing a direct connection between the medical device 502 and the mobile computing device 110. As shown in a state 702, the image capture device 116 of the mobile computing device 110 can capture an image of the QR code 504 as displayed on the medical device 502. The application 114, running on the mobile computing device 110, can receive a captured image of the QR code 504 from the image capture device 116. In certain implementations, the application 114 can be designed, programmed, or otherwise made available by a manufacturer of the medical device 502. In some examples, the application 114 can include its own set of security protocols configured to identify a user of the mobile computing device 110 and confirm that the user of the mobile computing device 110 has proper permissions and/or authorization to access the application 114. In such an example, the application 114 can process the information contained in the QR code 504 to determine if any encrypted data is included in the QR code 504.

As shown in a state 704 of FIG. 7, the application 114 can analyze the QR code 504 to determine both the encrypted dynamic data 508 as well as the encrypted static data 712. For example, as described above in regard to FIGS. 5 and 6, the QR code 504 can include both static and dynamic data. Referring back to FIG. 7, as shown in the state 704, the application 114 can utilize a decryption key 710 to decrypt the encrypted dynamic data 508 to generate dynamic data 506. Similarly, the application 114 can use the decryption key 710 to decrypt the encrypted static data 712 to generate decrypted static data 714. The processor of the mobile computing device 110 can use at least one of the dynamic data 506 and the static data 714 to establish a direct communication link between the mobile computing device 110 and the medical device 502. For example, as shown in a state 706, the processor of the mobile computing device 110 can use the connectivity information to establish a direct connection between the mobile computing device 110 and the medical device 502. In such an example, the communication data included in at least one of the decrypted dynamic data 506 and the decrypted static data 714 can include specific network information used by the processor of the mobile computing device 110 to establish the connection with the medical device 502. For example, the communication data can include a network identifier indicating a Wi-Fi or other similar wireless network to which the medical device 502 is to operably connect. Based upon this information, the processor of the mobile computing device 110 can operably connect to the same wireless network as the medical device 502. The communication data can also include a device ID associated with the medical device 502. Based upon the device ID, the processor of the mobile computing device 110 can establish a direct connection with the medical device 502 once connected to the same wireless network. Additionally, the communication data can include security and/or authentication information associated with establishing a direct connection with the medical device 502 including, for example, a username/password combination to be used by the processor of the mobile computing device 110 to establish a direct connection with the medical device 502.

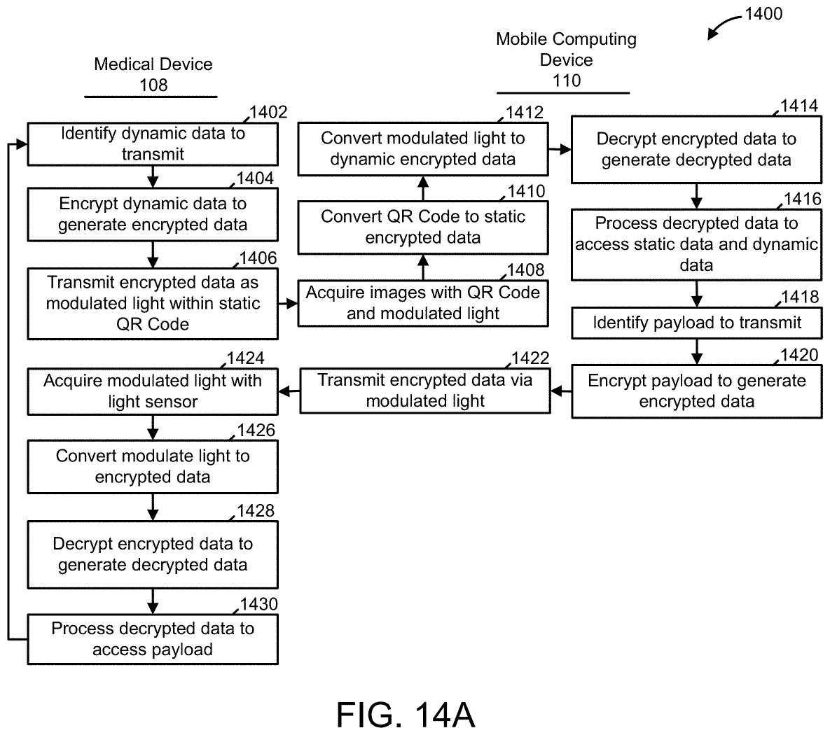

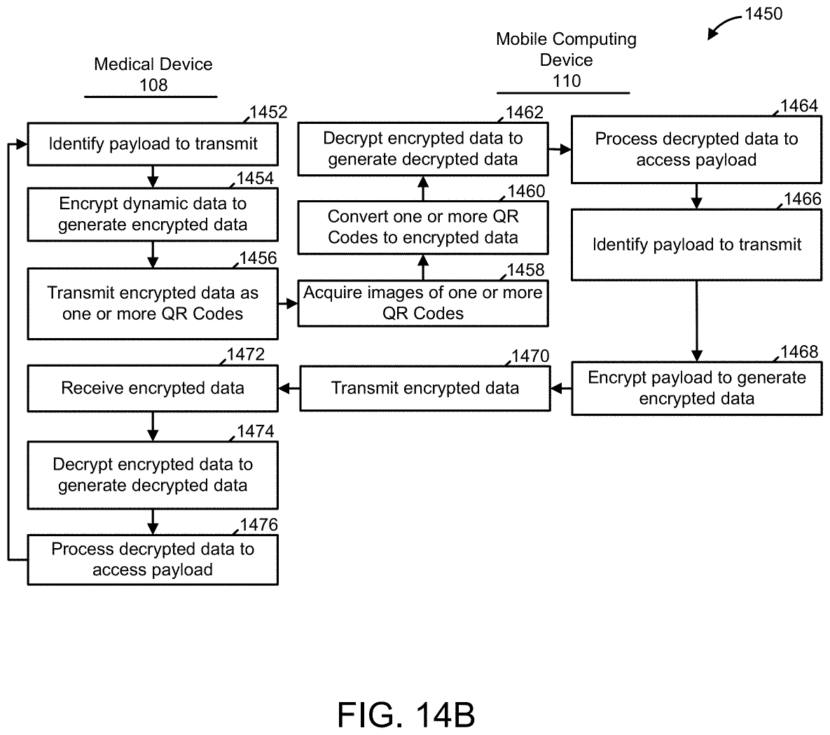

In some examples, in addition to using a QR code to transmit information, a mobile computing device can include a light transmitter configured to transmit a series of pulsed light including communication information directly to a medical device. For example, as shown in the state 706 in FIG. 7, the mobile computing device 110 can include a light emitter 716 configured to emit, for example, a pulsed and/or otherwise modulated series of light flashes that are detected by, for example, a light detector 718 integrated into the medical device 502. As such, the medical device 502 can receive information directly from the mobile computing device 110 via the light emitted by light emitter 716 and detected by light detector 718. Additional information related to transmitting information via modulated light as shown in the state 706 of FIG. 7 is described in additional detail in the discussion of FIG. 14A below.

In some examples, rather than establish a connection via an additional wireless network, the medical device 502 can be configured to provide for direct connections with user devices such as the mobile computing device 110. In such an example, the medical device 502 can be configured to include its own network ID and connection information in the QR code 504 such that, upon decryption of the information, the processor of the mobile computing device 110 can establish a direct connection with the medical device 502 via, for example, a local area network broadcast by the medical device 502. For example, the medical device 502 can be configured to establish direct connection with the user device such as the mobile computing device 110 via a Bluetooth connection, a mesh network connection, a local area network connection, a Wi-Fi connection, or other similar wireless connections configured to establish a direct connection between two communication devices.

Once the connection is established between the medical device 502 and the mobile computing device 110, information can be directly communicated between the medical device 502 and the mobile computing device 110. For example, as shown in a state 708 of FIG. 7, the medical device 502 can receive encrypted data 722 from the mobile computing device 110. For example, the mobile computing device 110 can output information using modulated light emitted by light emitter 716. The medical device 502 can detect the modulated light at light sensor 718 and process the modulated light. As further shown in the state 708, the processor of the medical device 108 can use decryption key 720 to decrypt the encrypted data 722 received from the mobile computing device 110 to generate decrypted data 724. The processor of the medical device 108 can be further configured to extract or otherwise determine instructions and data 726 from the decrypted data 724. In such an example, direct communication between the medical device 502 and the mobile computing device 110 can be quickly established while maintaining a secure communication link between the two devices by using a direct connection technique such as modulated light communication and/or infrared light communication.

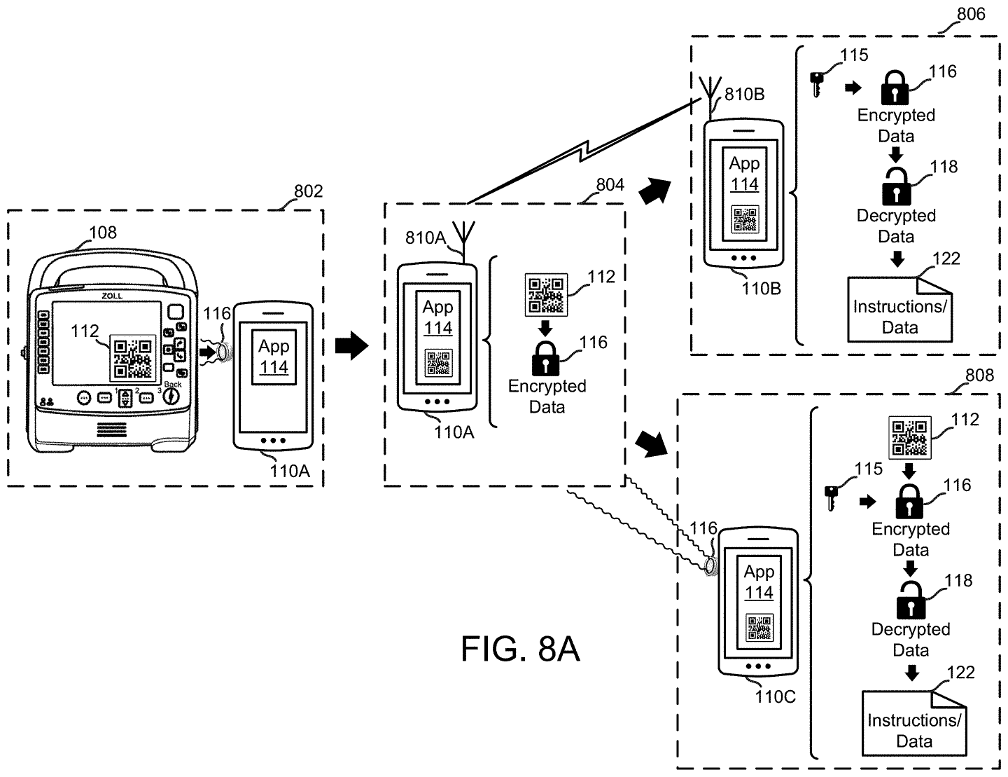

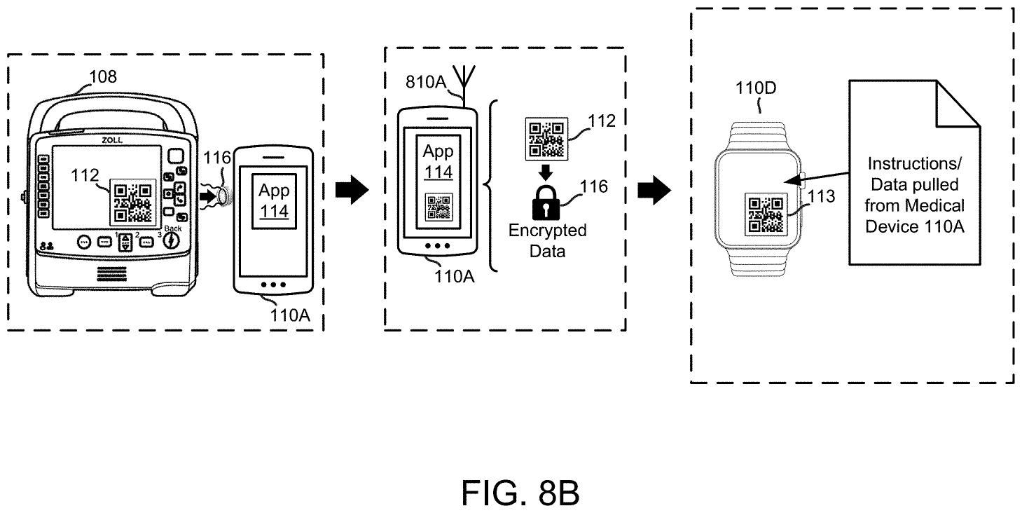

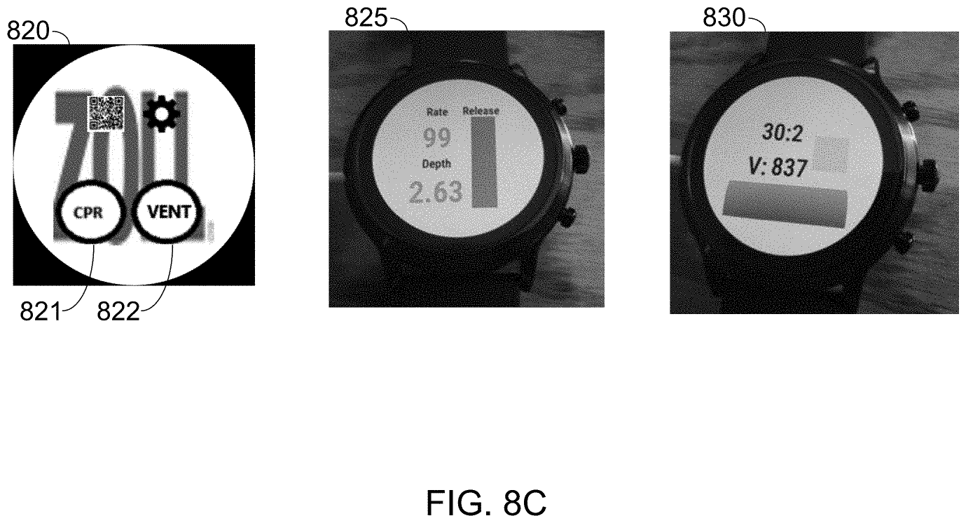

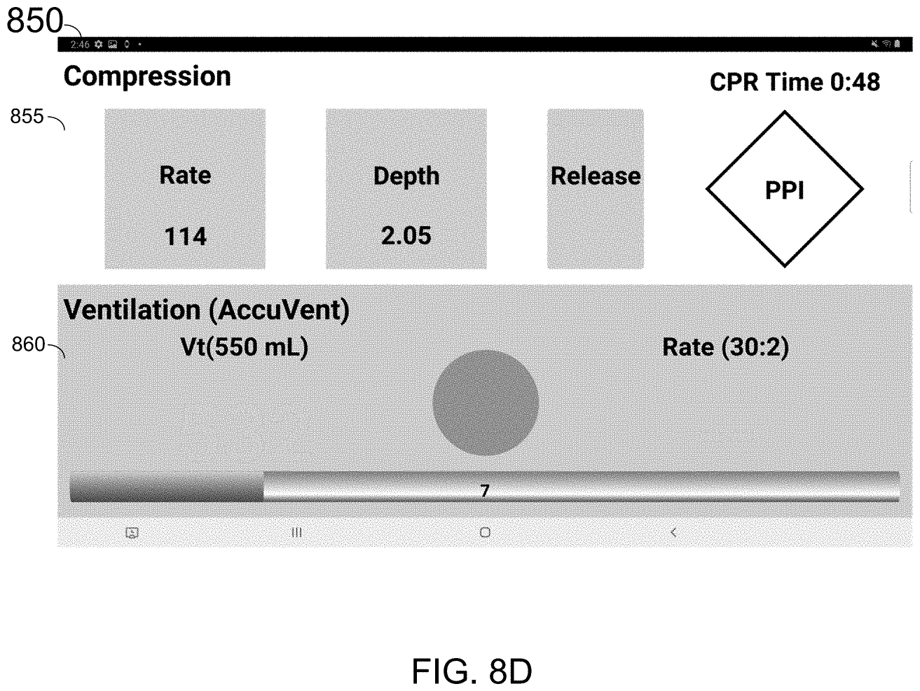

In certain implementations as described herein, a mobile computing device can be used as an intermediary and/or gateway device to establish a connection between a medical device and an additional computing device. For example, as shown in FIGS. 8A and 8B, an intermediary device can be used to establish a connection with another mobile device. For example, as shown in FIG. 8A, an intermediary device such as mobile computing device 110A can be used to establish a connection between medical device 108 and additional trusted mobile computing devices 110B and 110C. As shown in FIG. 8A, in a state 802, the mobile computing device 110 can establish a connection with the medical device 108 using, for example, the processes as described above in, for example, the description of FIG. 7. As shown in FIG. 8A, a processor of the mobile computing device 110 can cause the image capture device 116 to capture an image of the QR code 112 as displayed by the medical device 108A. As further shown in a state 804, the processor of the mobile computing device 110A can process the QR code 112 using, for example, the application 114 as described herein. The processor of the mobile computing device 110A can decode the QR code 112 to determine the encrypted data 118 contained within the QR code 112. The processor of the mobile computing device 110A can transmit a copy of the encrypted data 118 via a direct or indirect connection to, for example, a secondary mobile computing device 110B as shown in a state 806. The processor of the mobile computing device 110B can receive the encrypted data 118 from the mobile computing device 110A and process the encrypted data 118 using a copy of the application 114. The application can decrypt the encrypted data 118 using the decryption key 115 to generate the decrypted data 120. The processor of the mobile computing device 110B can further process the decrypted data 120 to generate the set of instructions/data 122 as described herein.

In some examples, the processor of the mobile computing device 110A can be further configured to decrypt the encrypted data 118 at state 804 using, for example, the decryption key 115, to generate the decrypted data 118 at state 804. In such examples, the processor of the mobile computing device 110A can forward the decrypted data to mobile computing devices 110B and/or 110C for further processing as described herein.

As further shown in FIG. 8A, the processor of the mobile computing device 110A, as shown in the state 804, can further be configured to display a copy of the QR code 112 within the application 114. In such an example, a processor of an additional secondary mobile computing device 110C can cause an image capture device 116 to capture an image of the QR code 112 as displayed by the mobile computing device 110A. For example as shown in a state 808, the processor of the secondary mobile computing device 110C can process the QR code 112 using, for example, the application 114 as described herein. The application 114 can use the decryption key 115 to decrypt the encrypted data 118 as decoded from the QR code 112 to generate the decrypted data 120. The processor of the mobile computing device 110C can further process the decrypted data 120 to determine the instructions/data 122 as described herein.

In such an example as illustrated in FIG. 8A, the process as shown provides for the ability to connect a mobile computing device that, for example, does not include an image capture device such as the example shown in the state 806. In such an example, secondary mobile computing device 110B can establish a connection with, for example, the medical device 108 using the instructions/data 122 decrypted from the information received from the mobile computing device 110A. Alternatively, as described above, one or both of secondary mobile computing devices 110B and 110C can be configured to pull or otherwise directly receive the decrypted data 118 directly from the mobile computing device 110A.

In some examples, the medical device 108 can enter a low power and/or sleep mode. In such an example, a primary mobile computing device such as mobile computing device 110A can be configured to wake the medical device 108 using, for example, an optical output such as a particular pattern of light. Upon awakening, the medical device 108 can be configured to display the optical code information for establishing a connection between the medical device 108 and the mobile computing device 110A. In another example, the information for waking the medical device 108 can be contained in a static QR code as described herein. Once awakened, the medical device 108 can be configured to display or otherwise output a dynamic and/or updated QR code for establishing a connection between the medical device 108 and the mobile computing device 110A as described herein.

It should be noted that both the secondary mobile computing devices 110B and 110C are shown as smartphones in FIG. 8A by way of example only. In implementation, each of the secondary mobile computing devices can be an alternative computing device such as, for example, a tablet computing device, a wearable computing device such as a smartwatch, a laptop computer, and other similar mobile computing devices. For example, as shown in FIG. 8B, a mobile computing device such as mobile computing device 110A can act as an intermediary device to establish a connection between the medical device 108 and a wearable computing device such as smartwatch 110D.

As shown in FIG. 8B, in order to establish a connection between the mobile computing device 110A and the smartwatch 110D, the smartwatch 110D can be configured to display an optical code such as QR code 113. The mobile computing device can be configured to capture and process an image of the QR code 113 and establish a direct connection with the smartwatch 110D. For example, the mobile computing device 110A can be configured to establish a Bluetooth connection with the smartwatch 110D such that information contained on the mobile computing device 110A can be transferred to and displayed on the smartwatch 110D. In such an example, a user and/or wearer of the smartwatch 110D can access information such as treatment and/or monitoring information collected by the medical device 108 via its connection with the mobile computing device 110A. As such, the data flow as shown in FIG. 8B is similar to that of 8A. However, rather than establish a direct connection with the medical device 108, the smartwatch 110D can connect to the mobile computing device 110A and use the mobile computing device 110A as an intermediary and/or gateway device to access information collected by the medical device 108. However, it should be noted that this arrangement is provided by way of example only and, in certain implementations, a wearable device such as smartwatch 110D can be configured to establish a direct connection with the medical device 108 to access treatment and/or monitoring information collected by the medical device 108.

FIGS. 8C and 8D illustrates sample screenshots showing information displayed by, for example, a mobile computing device as described herein. For example, FIG. 8C illustrates a set of screenshots showing information displayed on a smartwatch such as smartwatch 110D shown in FIG. 8B and described above. FIG. 8D illustrates a screenshot showing information displayed on a mobile computing device such as a smartphone, tablet, or other similar mobile computing device. The information shown in the screenshots as illustrated in FIGS. 8C and 8D can be received from, for example, a primary mobile computing device such as mobile computing device 110A as shown in FIGS. 8A and 8B and described above. In some examples, the information shown in the screenshots can be received directly from a medical device such as medical device 108 as shown in FIGS. 8A and 8B and described above.

For example, as shown in FIG. 8C, screenshot 820 illustrates an initial configuration screen displayed on, for example, a smartwatch as described herein. The initial configuration screen can include a user-selectable control 821 for viewing CPR related information as well as a user-selectable control 822 for viewing ventilation related information. As further shown in FIG. 8C, screenshot 825 includes information displayed on the smartwatch in response to a user selecting user-selectable control 821 to display CPR information. The CPR information as depicted in screenshot 825 can include, for example, compression rate information, compression depth information, compression release timing information (represented, for example, by a color-coded and dynamically sized bar), and other similar compression feedback information. In some examples, the compression rate information as illustrated in screenshot 825 can be color-coded, patterned, or otherwise dynamically updated to provide feedback to the user. For example, as the user provides chest compressions to a patient, the information in screenshot 825 can be color-coded based upon the user's performance. In certain implementations, the information can be green if the user is performing chest compressions to a preferred depth and at a preferred rate. If the user is not performing to a preferred level, the information can be yellow (e.g., if the user is within a certain percentage such as 10% of the preferred levels) or red (e.g., if the user is more than a certain percentage such as 10% away from the preferred levels). Screenshot 830 includes information displayed on the smartwatch in response to a user selecting user-selectable control 822 to display ventilation information. For example, the ventilation information can include ventilation rate information, tidal volume information, compression/ventilation ratio information (e.g., 30 compressions for every two ventilations), ventilation countdown information, and other similar ventilation feedback information. In some examples, like the compression rate information as illustrated in screenshot 825, the ventilation information as illustrated in screenshot 830 can be color-coded, patterned, or otherwise dynamically updated to provide feedback to the user. For example, as the user provides ventilation to the patient, the information in screenshot 835 can be color-coded based upon the user's performance. In certain implementations, the information can be green if the user is providing ventilation at a preferred rate. If the user is not providing ventilation at the preferred rate, the information can be yellow (e.g., if the user is within a certain percentage such as 10% of the preferred ventilation rate) or red (e.g., if the user is more than a certain percentage such as 10% away from the preferred ventilation rate). It should be noted that the CPR information and the ventilation information as shown screenshots 825 and 830 of FIG. 8C is provided by way of example only and can vary based upon the type of information collected by the medical device and the communication and display capabilities of the mobile computing device.

In certain implementations, the user of a mobile computing device can be assigned a particular role or security level that provides access to additional information for review and/or processing. As shown in FIG. 8D, a user of a mobile computing device can access a set of overall treatment information including both CPR and ventilation performance information. As shown in screenshot 850 of FIG. 8D, compression performance information 855 as well as ventilation performance information 860 can be displayed to, for example, a supervisor or other similar user with access and/or permission to review such information. As shown in screenshot 850, the compression performance information 855 can include, for example, compression rate information, compression depth information, release timing information, overall CPR timing (e.g., total time that CPR was administered to the patient), and other similar compression information. The ventilation information 860 can include, for example, ventilation rate information, tidal volume information, compression/ventilation ratio information, ventilation countdown information, and other similar ventilation information. Similar to the discussion of screenshots 825 and 820 of FIG. 8C above, the compression performance information 855 and/or the ventilation information 860 can be color-coded, patterned, or otherwise dynamically updated to provide feedback to the user. For example, in certain implementations, the compression performance information 855 and/or the ventilation information 860 can be green if the user is providing compressions and/or ventilation at a preferred rate. If the user is not providing the compressions and/or ventilation at the preferred rate, the information can be yellow (e.g., if the user is within a certain percentage such as 10% of the preferred compression and/or ventilation rate) or red (e.g., if the user is more than a certain percentage such as 10% away from the preferred compression and/or ventilation rate).

It should be noted that the compression rate and ventilation information as shown in FIGS. 8C and 8D and described above is described as being color-coded by way of example only. In certain implementations, the information can include additional dynamically updated performance information to provide feedback to the user of the mobile computing device. In some examples, additional feedback beyond dynamically updated visual feedback information can be provided. For example, one or more audible outputs can provide the user feedback information regarding their current performance as described herein.

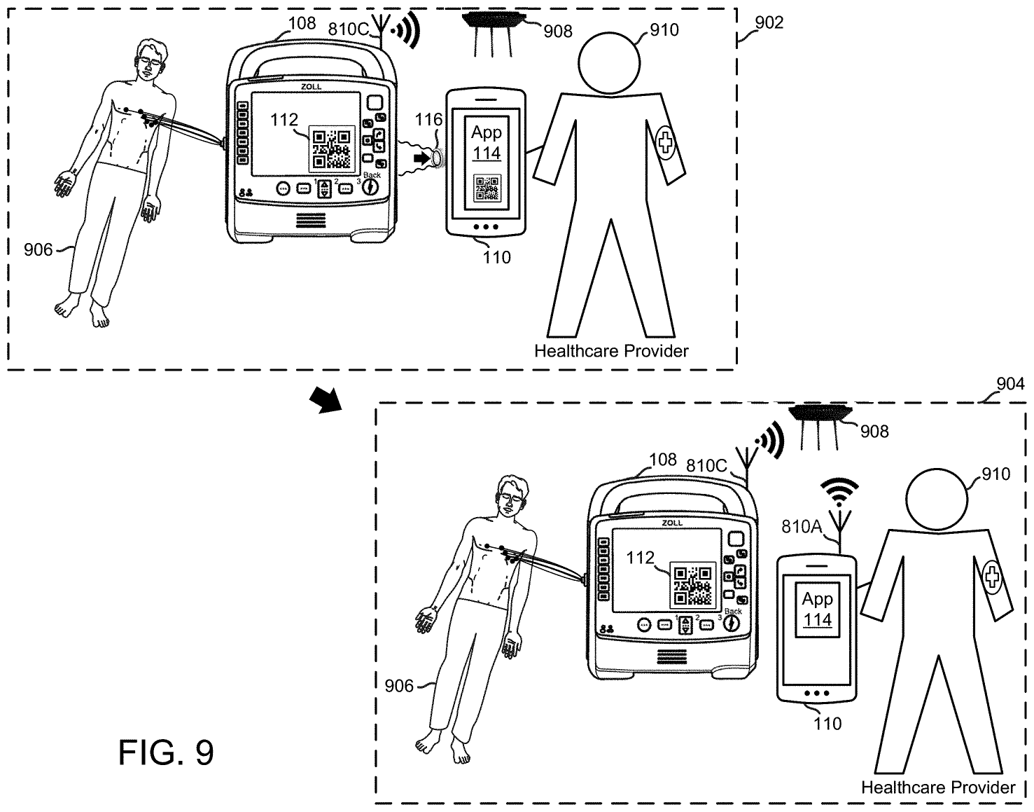

In certain examples, using the processes as described herein, information collected by a medical device can be provided to, for example, a health care provider and/or other similar caregiver providing medical treatment to a patient. As such, sensitive data collected by the medical device 108 during treatment of the patient can be securely transferred to a mobile computing device associated with the health care provider using the processes as described herein. For example FIG. 9 illustrates an example related to connecting a mobile computing device associated with a health care provider to a medical device providing treatment to a patient. As shown in a state 902 of FIG. 9, a patient 906 is being treated by the medical device 108. A health care provider 910 arrives on scene and wishes to connect their mobile computing device 110 to the medical device 108 to obtain information related to treatment of the patient 906. As further shown in the state 902, the medical device 108 is operably connected to a wireless network via a network access point 908. As further shown, an antenna 810C of the medical device 108 is configured to transmit and receive information from the wireless network via the access point 908. In order to obtain information from the medical device 108, the health care provider 910 can operatively connect their mobile computing device 110 to the access point 908 as well to obtain information from the medical device 108. As such, using the processes as described herein, the processor of the mobile computing device 110 can cause the image capture device 116 to capture an image of the QR code 112 as displayed by the medical device 108. Using a process similar to that as described above in, for example, FIG. 7, the processor of the mobile computing device 110 can decode the information contained within the QR code 112 using, for example, the application 114. The application 114 can be configured to decrypt any encrypted information contained within the decoded QR code 112 to determine any network identification and connectivity information contained within the encrypted information.

As further shown in a state 904 of FIG. 9, the mobile computing device 110 has established a direct communication link with the access point 908, communicating via information received by the antenna 810A of the mobile computing device. In such an example, the health care provider 910 can receive patient-specific treatment information from the medical device 108 on the mobile computing device 110 using the processes as described herein.

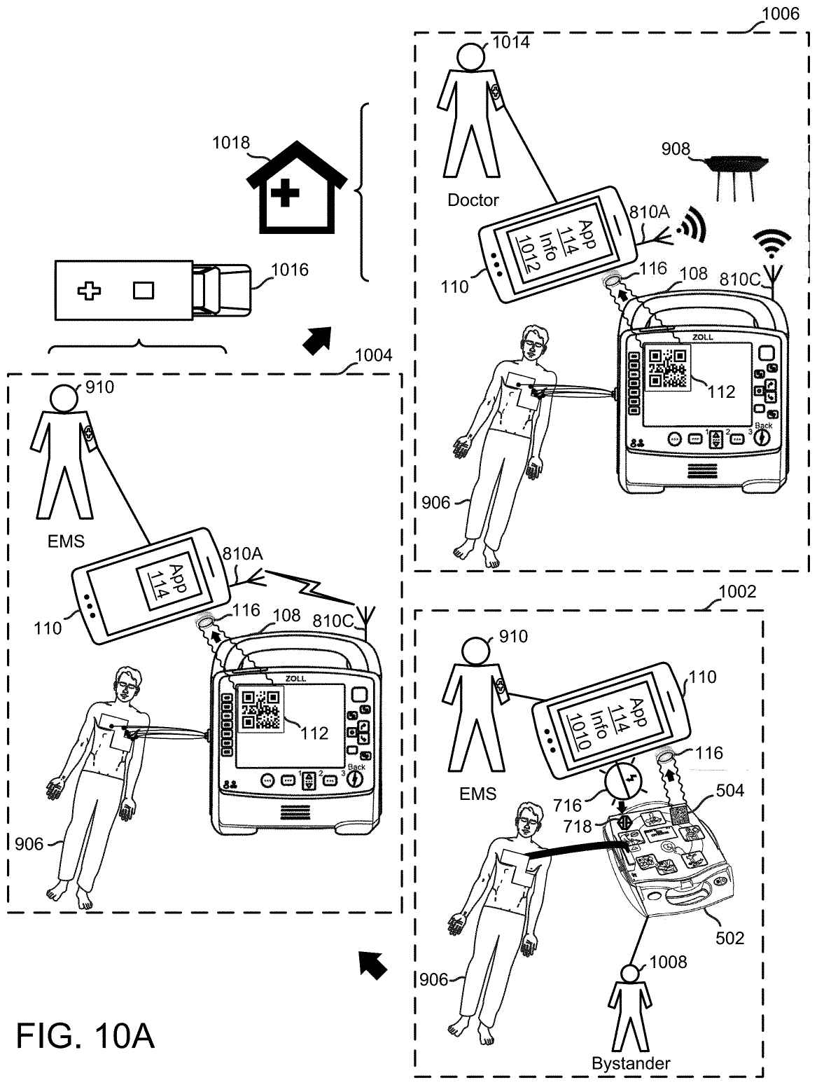

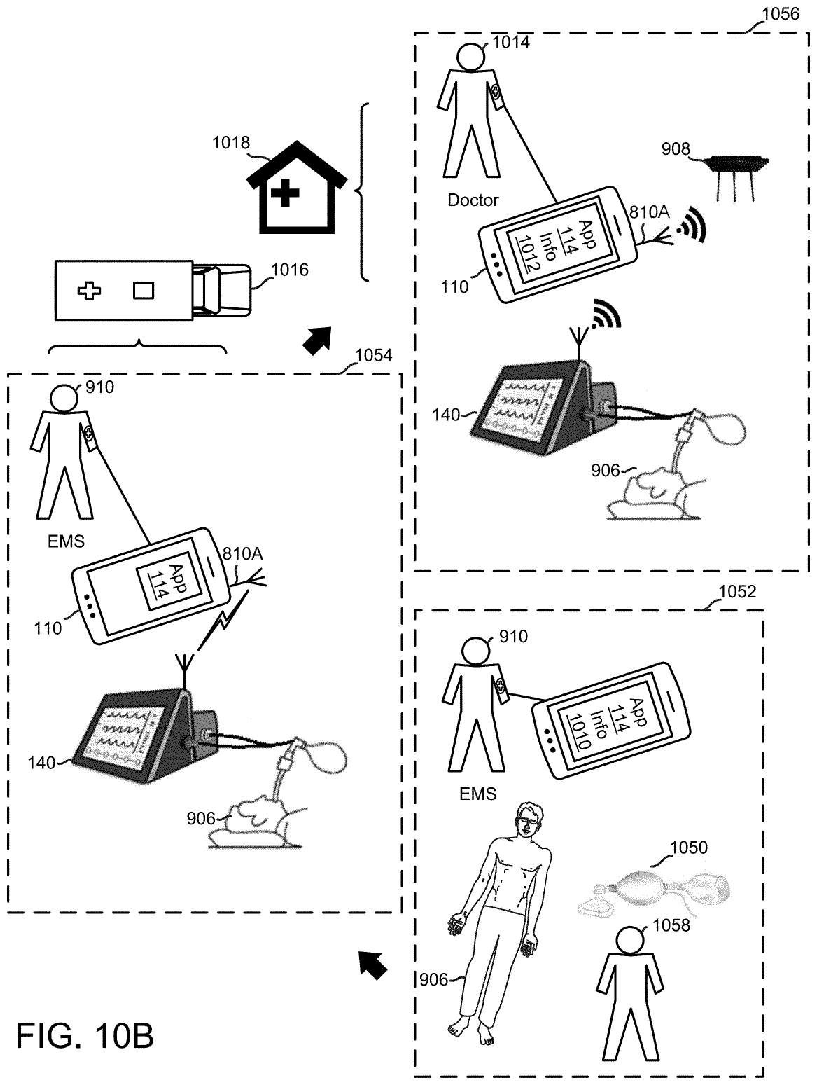

In some examples, the systems and processes described herein can be used to provide information related to a patient receiving treatment at various states in locations throughout the treatment process. For example, as shown in FIG. 10A, the systems and processes as described herein can be used to provide patient-specific information related to treatment provided to a patient by a medical device such as a defibrillator as the patient transitions from an initial site of an event requiring treatment until the patient ultimately arrives at the hospital. More specifically, as shown in FIG. 10A, in a state 1002, the patient 906 suffers an event that requires treatment by the medical device 502. As the patient 906 transitions to a state 1004, the patient 906 is transported by an ambulance 1016. As further shown in FIG. 10A, in a state 1006, the patient 906 arrives at a hospital 1018. Throughout each of the states as shown in FIG. 10A, patient-specific information can be transferred between medical devices and mobile computing devices such that the patient information associated with treatment provided to the patient is maintained throughout the patient's trip from the site of the initial event until the patient arrives at the hospital 1018.

As further shown in FIG. 10A, in the state 1002, the patient 906 experiences an adverse cardiac event that requires treatment of the patient by the medical device 502. As shown, a bystander 1008 can begin treatment of the patient 906 using the medical device 502. After a period of time, the health care provider 910 arrives and takes over treatment of the patient 906. In order to determine what treatment has already been provided to the patient 906, the health care provider 910 can operably connect their mobile computing device 110 to the medical device 502. For example, using processes as described herein, the image capture device 116 of the mobile computing device 110 can capture an image of the QR code 504. The application 114 can process the QR code 504 and, based upon encrypted information contained within the QR code 504, establish a direct connection between the mobile computing device 110 and the medical device 502. The application 114 can receive patient information 1010 regarding treatment of the patient 906 from the medical device 502 via the direct connection between the medical device 502 and the mobile computing device 110.

As further shown in FIG. 10A, in the state 1004, the health care provider 910 has transitioned the patient 906 into the ambulance 1016. As such, treatment of the patient 906 is now being provided by the medical device 108. If not already connected, the health care provider 910 can operably connect their mobile computing device 110 to the medical device 108 using, for example, the process as described above in reference to, for example, FIGS. 7 and 8. Once a connection is established between the mobile computing device 110 and the medical device 108, the patient information 1010 as received from the medical device 502 can be transferred to the medical device 108 such that the medical device 108 has updated treatment information regarding treatment already provided to the patient 906. As the ambulance 1016 transports the patient 906 to the hospital 1018, the mobile computing device 110 can continue to receive updated statistical information regarding treatment of the patient 906 from the medical device 108.

As shown in the state 1006 of FIG. 10A, once the patient 906 arrives at the hospital 1018, a second health care provider such as a doctor 1014 can begin treatment of the patient 906. In order to provide the doctor 1014 with updated treatment information regarding treatment of the patient 906 up to this point, the doctor 1014 can connect her mobile computing device 110 to the medical device 108 using, for example, the processes as described herein. Once a connection between the mobile computing device 110 and the medical device 108 is established, the doctor 1014 can receive updated patient information 1012 about the treatment already provided to the patient 906. Based upon this information 1012, the doctor 1014 can continue treatment of the patient 906 while considering what treatment the patient 906 has already received and the patient's response to the previously provided treatment.

In a second example, as shown in FIG. 10B, the systems and processes as described herein can be used to provide patient-specific information related to treatment provided to a patient by a second medical device such as a ventilator or other respiratory treatment device as the patient transitions from an initial site of an event requiring treatment until the patient ultimately arrives at the hospital. More specifically, as shown in FIG. 10B, in a state 1052, the patient 906 suffers an event that requires treatment by the medical device 1050. As the patient 906 transitions to a state 1054, the patient 906 is transported by an ambulance 1016. As further shown in FIG. 10B, in a state 1056, the patient 906 arrives at a hospital 1018. Throughout each of the states as shown in FIG. 10B, patient-specific information can be transferred between medical devices and mobile computing devices such that the patient information associated with treatment provided to the patient is maintained throughout the patient's trip from the site of the initial event until the patient arrives at the hospital 1018.

As further shown in FIG. 10B, in the state 1052, the patient 906 experiences an adverse respiratory event that requires treatment of the patient by the medical device 1050. As shown, a first responder 1058 can begin treatment of the patient 906 using the medical device 1050. After a period of time, the health care provider 910 arrives and takes over treatment of the patient 906. In order to determine what treatment has already been provided to the patient 906, the health care provider 910 can operably connect their mobile computing device 110 to the medical device 1050. For example, using processes as described herein, the image capture device 116 of the mobile computing device 110 can capture an image of a QR code associated with the medical device 1050. The application 114 can process the QR code and, based upon encrypted information contained within the QR code, establish a direct connection between the mobile computing device 110 and the medical device 1050. The application 114 can receive patient information 1010 regarding treatment of the patient 906 from the medical device 1050 via the direct connection between the medical device 1050 and the mobile computing device 110.

As further shown in FIG. 10B, in the state 1054, the health care provider 910 has transitioned the patient 906 into the ambulance 1016. As such, treatment of the patient 906 is now being provided by the medical device 140. If not already connected, the health care provider 910 can operably connect their mobile computing device 110 to the medical device 140. Once a connection is established between the mobile computing device 110 and the medical device 140, the patient information 1010 as received from the medical device 502 can be transferred to the medical device 140 such that the medical device 140 has updated treatment information regarding treatment already provided to the patient 906. As the ambulance 1016 transports the patient 906 to the hospital 1018, the mobile computing device 110 can continue to receive updated patient information regarding treatment of the patient 906 from the medical device 140.

As shown in the state 1056 of FIG. 10B, once the patient 906 arrives at the hospital 1018, a second health care provider such as a doctor 1014 can begin treatment of the patient 906. In order to provide the doctor 1014 with updated treatment information regarding treatment of the patient 906 up to this point, the doctor 1014 can connect her mobile computing device 110 to the medical device 140 using, for example, the processes as described herein. Once a connection between the mobile computing device 110 and the medical device 140 is established, the doctor 1014 can receive updated patient information 1012 about the treatment already provided to the patient 906. Based upon this information 1012, the doctor 1014 can continue treatment of the patient 906 while considering what treatment the patient 906 has already received and the patient's response to the previously provided treatment.

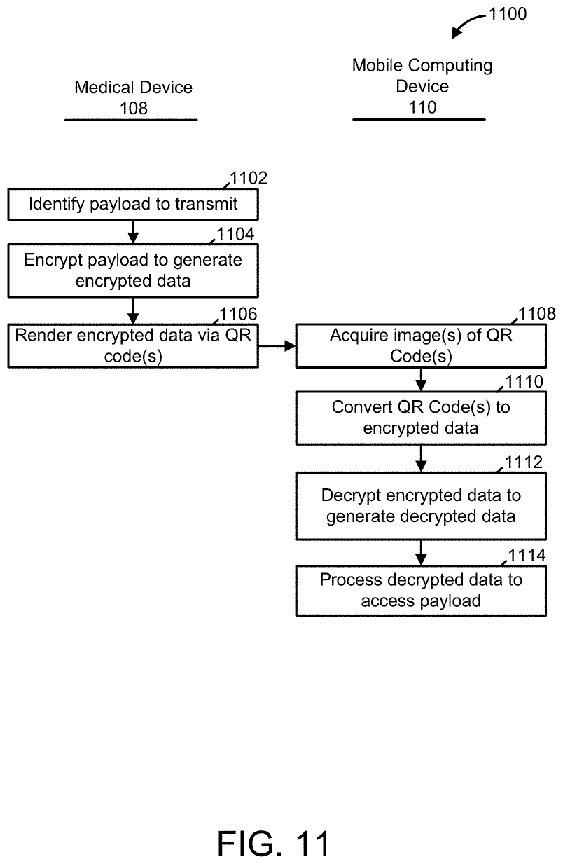

It should be noted that the system and hardware configuration examples as shown in FIGS. 1-10B are provided by way of example only. More specific process details regarding transfer of information, decoding of QR codes, decrypting of encrypted information, and processing the decrypted information is provided in the following process flow descriptions of FIGS. 11-17.

For example, FIG. 11 illustrates a process flow 1100 describing accessing information displayed by a medical device in a QR code by a mobile computing device similar to the system as illustrated in FIG. 1 and described above. More specifically, as shown in the process 1100, a processor associated with a medical device, such as medical device 108, can identify 1102 a payload and/or set of information to transmit or otherwise convey to another computing device. The processor can encrypt 1104 the identified payload to generate a set of encrypted data. For example, the processor can use an encryption key stored on the medical device to encrypt the payload. The processor can further render 1106, or otherwise encode, the encrypted data as one or more QR codes as described herein such that the QR codes represent the encoded and encrypted data. The processor of the medical device can further cause the medical device to display the one or more rendered QR codes such that another computing device can access at least a portion of the QR codes.

As further shown in FIG. 11, the process 1100 continues with functions performed by a mobile computing device such as mobile computing device 110 as shown in FIG. 1 and described above. A processor of the mobile computing device can acquire 1108 one or more images of the QR codes as displayed by the medical device. For example, the processor can cause an image capture device associated with or otherwise integrated into the mobile computing device to capture an image of the displayed QR codes. The processor of the mobile computing device can further convert 1110 the QR codes to determine the encrypted data. For example, the processor of the mobile computing device can use a QR code decoding algorithm or other similar process to decode the information contained within the QR code to determine the encrypted data. The processor of the mobile computing device can further decrypt 1112 the encrypted data to generate a set of decrypted data. For example, as described herein, the processor can access an application on the mobile computing device that includes a decryption key configured to decrypt the encrypted data contained within the QR code. The processor of the mobile computing device can further process 1114 the decrypted data to access the payload information included in the QR code as encrypted by the processor of the medical device.

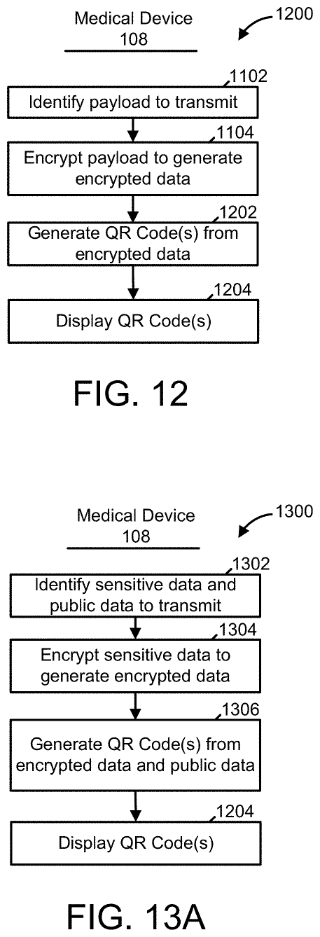

FIG. 12 illustrates process 1200, a more detailed process as performed by, for example, a processor of a medical device such as medical device 108 as described herein. For example, the processor can identify 1102 a payload to transmit and encrypt 1104 the payload to generate encrypted data, similar to the process 1100 as described above in the discussion of FIG. 11. As further shown in FIG. 12, the processor of the medical device can be configured to generate 1202 one or more QR codes from the encrypted data. For example, the processor can use QR code encoding techniques and/or related algorithms to generate 1202 one or more QR codes representative of the encrypted data. The processor of the medical device can further be configured to display 1204 the one or more QR codes via, for example, a display on the medical device as described herein.

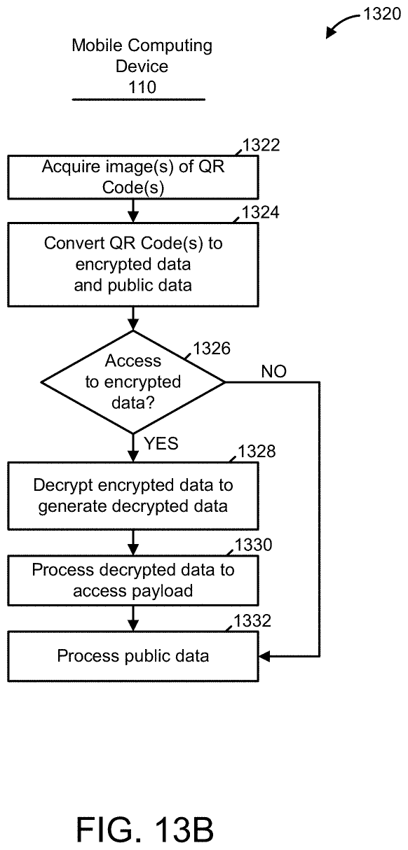

As described above in, for example, the discussion of FIG. 4, a QR code as described herein can include both sensitive data as well as publicly-accessible data. A process 1300 as shown in FIG. 13A is directed toward generating one or more QR codes including both sensitive data and public data for transmission or display to another computing device such as a mobile computing device as described herein. For example, as shown in process 1300A, a processor of a medical device, such as medical device 108 as described herein, can be configured to identify 1302 sensitive data and public data for transmission to another computing device. The processor of the medical device can be further configured to encrypt 1304 the sensitive data to generate encrypted sensitive data. The processor can combine the encrypted sensitive data along with the public data and generate 1306 one or more QR codes including both the encrypted sensitive data and the public data. The processor of the medical device can be further configured to display 1204 the one or more QR codes via, for example, a display on the medical device as described herein.

In certain implementations, a mobile device that is not authorized to access the sensitive encrypted information contained within a visual indicator such as the optical QR codes as described herein can attempt to access other types of information contained within the visual indicator. For example, as described above in the discussion of FIG. 13A, an optical code such as a QR code can include both encrypted sensitive data that is accessible only through appropriate authorization and/or decryption schemes as well as publicly accessible data. In such an example, when a mobile device that is not authorized to access the encrypted sensitive information attempts to process the optical code, the device can only display or otherwise access information contained within or related to the publicly accessible information. Such publicly accessible information may include, for example, information about the type of medical device, company information, medical guidance for how to use the medical device, and/or other relevant information that would be helpful the general public. The visual indicator may provide a link to an internet site or associated app for download that contains the publicly accessible information in a presentable format, or may provide the publicly accessible information directly to the device. In some cases, the medical guidance for how to use the medical device may be interactive in querying the user as to the nature of the medical emergency and providing instructions, suggestions, or other types of guidance for how to treat the medical emergency. In some embodiments, the publicly accessible information may also provide the ability for the user to call or otherwise contact emergency services personnel for further assistance.

For example, as further shown in FIG. 13B, a process 1320 can be directed to determining which data a mobile computing device, such as mobile computing device 110, has access to and processing the data contained within an optical code such as a QR code accordingly. A processor of the mobile computing device can acquire 1322 one or more images of the QR codes as displayed by the medical device. For example, the processor can cause an image capture device associated with or otherwise integrated into the mobile computing device to capture an image of the displayed QR codes. The processor of the mobile computing device can further convert 1324 the QR codes to determine the data contained within the QR code. As described herein, the data contained within the QR code can include both encrypted data as well as publicly accessible data, as discussed herein. For example, the processor of the mobile computing device can use a QR code decoding algorithm or other similar process to decode the information contained within the QR code to determine both the encrypted data as well as the publicly accessible data.

As further shown in FIG. 13B, the processor of the mobile computing device can determine 1326 whether the mobile computing device (or, more specifically, the user of the mobile computing device) has access to the encrypted data. To determine 1326 access, the processor can determine whether the user is authorized to access the encrypted data or has otherwise been authenticated and approved to access the sensitive data as described herein. If the processor of the mobile computing device does determine 1326 that the user has access to the encrypted data, the processor can further decrypt 1328 the encrypted data to generate a set of decrypted data. For example, as described herein, the processor can access an application on the mobile computing device that includes a decryption key configured to decrypt the encrypted data contained within the QR code. The processor of the mobile computing device can further process 1330 the decrypted data to access the payload information included in the QR code as encrypted by the processor of the medical device as well as process 1332 the public data included in the QR code.

Conversely, if the processor of the mobile computing device does not determine 1326 that the user of the mobile computing device has access to the encrypted data, the processor can proceed to process 1332 the publicly accessible data as contained within the QR code. In such an example, the publicly accessible data can include additional information related to the medical device displaying the QR code (e.g., manufacturer information, operating instructions, a link or other similar directions to a website or other similar resource including guidance for dealing with emergency situations) as well as additional resources for a bystander that may be witnessing or otherwise assisting with a medical emergency such as emergency medical assistance contact information. For example, if the medical device displaying the QR code is an AED, the public information can include instructions for positioning one or more therapy electrodes on the patient, instructions for how to provide therapy shocks to the patient in response to detection of a cardiac arrythmia, contact information for the local EMTs, information related to the closest hospital or other treatment facility, and other general knowledge that would be beneficial in helping the user of the medical device.

As described herein, in certain implementations, bidirectional communication between both the mobile computing device and a medical device is provided. For example, information contained within a QR code as displayed by a medical device can be used by a mobile computing device to establish a connection between the mobile computing device and the medical device. In certain implementations, information can be transmitted from the mobile computing device to a medical device using, for example, modulated light as described above in regard to FIG. 7. FIG. 14A illustrates a sample process 1400 that provides detail regarding bidirectional communication between a medical device such as medical device 108 and a mobile computing device such as mobile computing device 110.

More specifically, as shown in FIG. 14A, a processor of a medical device can be configured to identify 1402 dynamic data to transmit to a mobile computing device. The processor of the medical device can be configured to encrypt 1404 the dynamic data to generate encrypted data. The processor of the medical device can be further configured to transmit 1406 the encrypted data as modulated light within a static QR code. For example, as shown in FIGS. 5 and 6 and described above, a QR code can include both a static portion as well as a dynamic portion represented by, for example, modulated light as described herein.

Referring again to FIG. 14A, a processor of the mobile computing device can be configured to acquire 1408 images of the QR code including the modulated light as described herein. The processor of the mobile computing device can convert 1410 the QR code to extract the static encrypted data contained within the QR code. The processor of the mobile computing device can further be configured to convert 1412 the modulated light to decode the dynamic encrypted data contained within the QR code. The processor of the mobile computing device can decrypt 1414 the encrypted data to generate decrypted data using, for example, a decryption key as described herein. The processor of the mobile computing device can process 1416 the decrypted data to access the static data and the dynamic data contained within the decrypted data. The processor of the mobile computing device can further identify 1418 payload and/or other similar information to transmit back to the medical device. The processor of the mobile computing device can encrypt 1420 the payload to generate encrypted data using, for example, an encryption key stored on the mobile computing device. The processor of the mobile computing device can transmit 1422 the encrypted data via modulated light using, for example, a light emitter as described above in regard to FIG. 7.

As further shown in FIG. 14A, the processor of the medical device can acquire 1424 the modulated light using a light sensor or other similar light detector integrated into or otherwise associated with the medical device. The processor of the medical device can convert 1426 the modulated light to determine the encrypted data as transmitted by the mobile computing device by demodulating the received light. The processor of the medical device can decrypt 1428 the encrypted data using, for example, a decryption key stored in the medical device to generate decrypted data. The processor of the medical device can process 1430 the decrypted data to access the payload as transmitted by the mobile computing device. Based upon the payload, the processor of the medical device can identify 1402 additional dynamic data to transmit to the mobile computing device, and at least a portion of the process 1400 can repeat as shown in FIG. 14A.

As further described herein, a direct connection using a wireless communication protocol can be established between a medical device such as medical device 108 and a mobile computing device such as mobile computer device 110. For example, as shown in FIG. 14B, a process 1450 includes bidirectional communication between a medical device and a mobile computing device using a wireless communication link as described herein. More specifically, as shown in FIG. 14B, a processor of the medical device can identify 1452 a payload to transmit to a mobile computing device. The processor can encrypt 1454 dynamic data contained within the payload to generate encrypted data. The processor of the medical device can transmit 1456 the encrypted data as one or more QR codes as described herein. In certain implementations, the processor of the medical device can process the encrypted data and generate the one or more QR codes for subsequent scanning and decryption by, for example, a mobile computing device as described herein.

As further shown in FIG. 14B, a processor of the mobile computing device can acquire 1458 one or more images of the one or more QR codes. The processor of the mobile computing device can convert 1460 the one or more QR codes to determine encrypted data contained within the QR codes. The processor of the mobile computing device can decrypt 1462 the encrypted data using, for example, a decryption key to generate decrypted data and process 1464 the decrypted data to access the payload contained within the decrypted data. The processor of the mobile computing device can further identify 1466 a payload or other information to transmit to the processor of the medical device. The processor of the mobile computing device can encrypt 1468 the payload to generate encrypted data and transmit 1470 the encrypted data to the processor of the medical device.

As further shown in FIG. 14B, the processor of the medical device can receive 1472 the encrypted data. The processor of the medical device can further process the encrypted data to decrypt 1474 the encrypted data using, for example, a decryption key stored within the medical device. The processor of the medical device can further process 1476 that encrypted data to access the payload contained within the data is transmitted from the mobile computing device and process the payload. Based upon the payload, the processor of the medical device can identify additional information to transmit to the mobile computing device, and at least a portion of the process 1450 as shown in FIG. 14B can repeat.

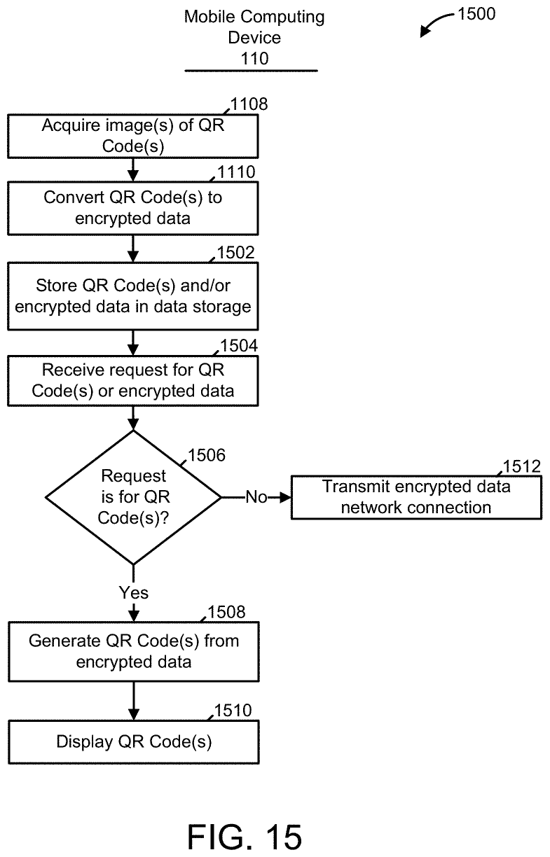

As noted above in, for example, the discussion of FIGS. 8A and 8B, a mobile computing device can be used as a gateway device to establish connections between additional mobile computing devices and, for example, a medical device as described herein. FIG. 15 illustrates a sample process 1500 related to configuring a mobile computing device such as mobile computing device 110 to function as a gateway device as described herein. For example, a processor of the mobile computing device can acquire 1108 one or more images of one or more QR codes as displayed by a medical device. The processor of the mobile computing device can convert 1110 the QR codes to encrypted data by, for example, decoding the QR code information. The processor of the mobile computing device can further store 1502 the QR codes and/or the encrypted data in a local data storage on the mobile computing device. The processor of the mobile computing device can further receive 1504 a request for QR codes and/or encrypted data from another mobile computing device such as a secondary mobile computing device as shown in FIG. 8A and described above. The processor of the mobile computing device can determine 1506 whether the request is for a QR code as stored in the memory. If the processor of the mobile computing device determines 1506 that the request is for a QR code, the processor can generate 1508 a QR code from the stored encrypted data and display 1510 the QR code. Alternatively, if the processor determines 1506 request is not for a QR code the processor of the mobile computing device can transmit 1512 encrypted data network connection information to the requesting mobile computing device.

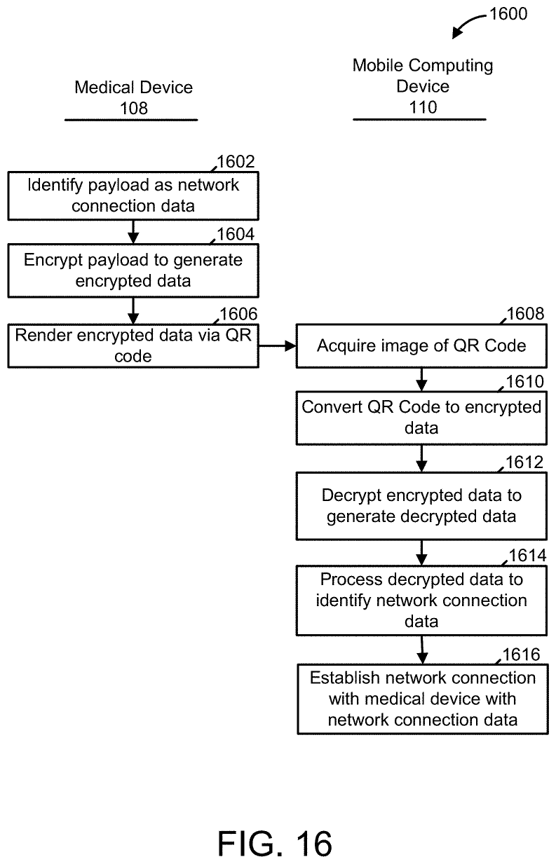

Additionally, as described herein, a mobile computing device can use information contained within the QR code to establish a communication link with a medical device. For example, as described above in FIG. 1, information contained within a QR code can be used to establish connection between, in certain implementations, the mobile computing device 110 and the medical device 108. FIG. 16 illustrates sample process 1600 for establishing a connection between a medical device such as medical device 108 and a mobile computing device such as mobile computing device 110.

As shown in FIG. 16, a processor of the medical device can identify 1602 payload information including network connection data. The processor of the medical device can encrypt 1604 the payload to generate encrypted data, the encrypted data including the network connection data. The processor of the medical device can further render 1606 the encrypted data via QR code for display to, for example, a mobile computing device.

As further shown in FIG. 16, a processor of the mobile computing device such as mobile computing device 110 can acquire 1608 an image of the QR code as displayed by, for example, the medical device. The processor of the mobile computing device can convert 1610 the QR code to determine the encrypted data contained therein. The processor of the mobile computing device can decrypt 1612 the encrypted data to generate decrypted data and process 1614 the decrypted data to identify the network connection data as provided by the medical device. The processor of the mobile computing device can establish 1616 a network connection with the medical device using the network connection data as decrypted from the encrypted information contained within the QR code. For example, the processor of the mobile computing device can establish a connection between a network interface of the mobile computing device and a wireless network identified in the network connection data. The processor of the mobile communication device can further identify the medical device on the network by searching for a device ID associated with the medical device and included in the network communication data. Once identified on the wireless network, the processor of the mobile computing device can establish a connection with the medical device as described herein.

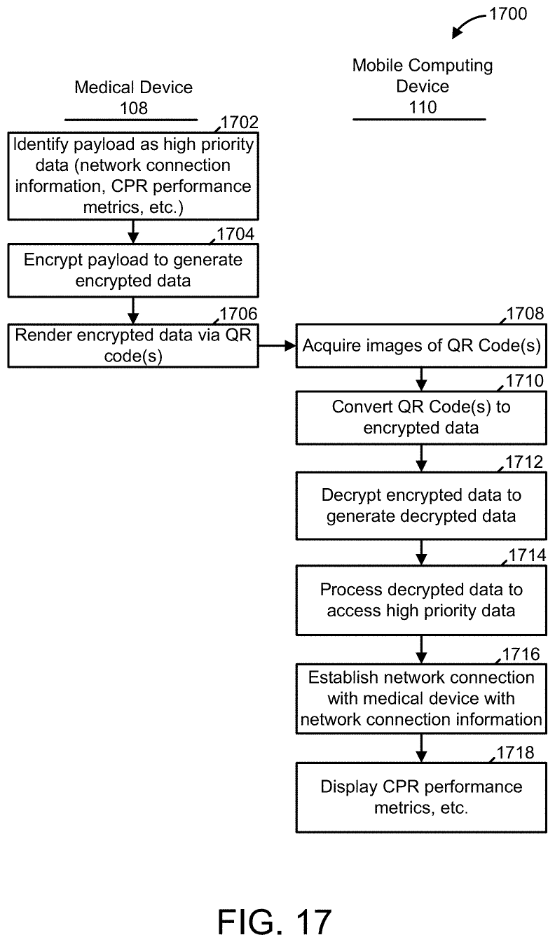

In another example as described herein, for example as shown in FIGS. 10A and 10B, and described above, a medical device can provide patient information related to the treatment of a patient to a mobile computing device. For example, FIG. 17 illustrates a process 1700 that includes transmitting patient information from a medical device such as medical device 108 to a mobile computing device such as mobile computing device 110 as described herein.

As shown in FIG. 17, a processor of the medical device can identify 1702 a payload as high priority data including, for example, network connection information, CPR performance metrics, defibrillator treatment information, ventilator information, medical device self-test information, and other patient information related to treatment of the patient.

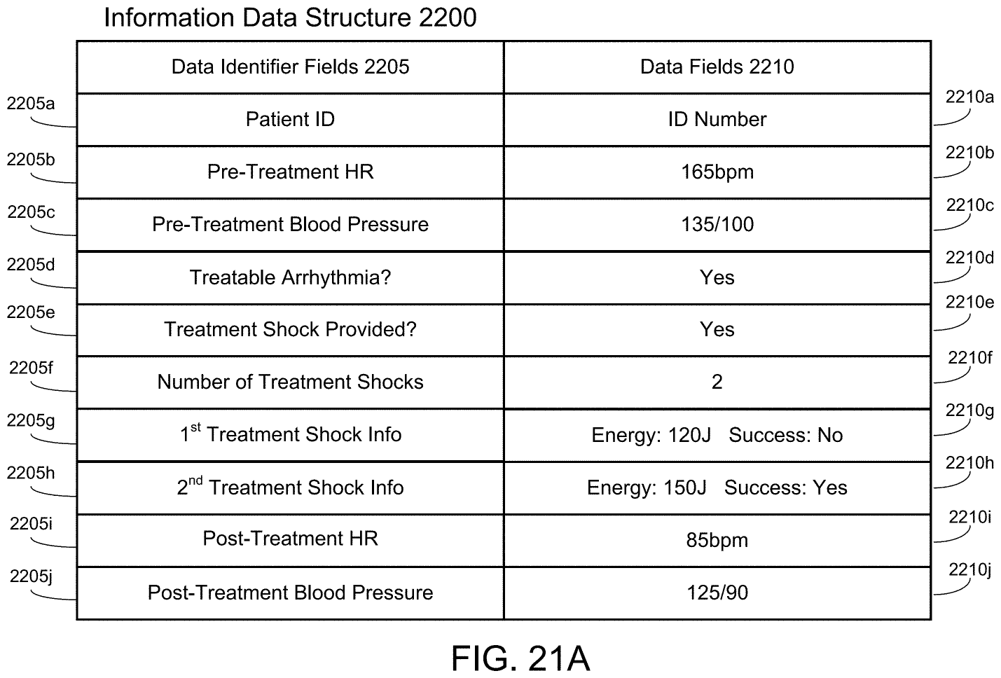

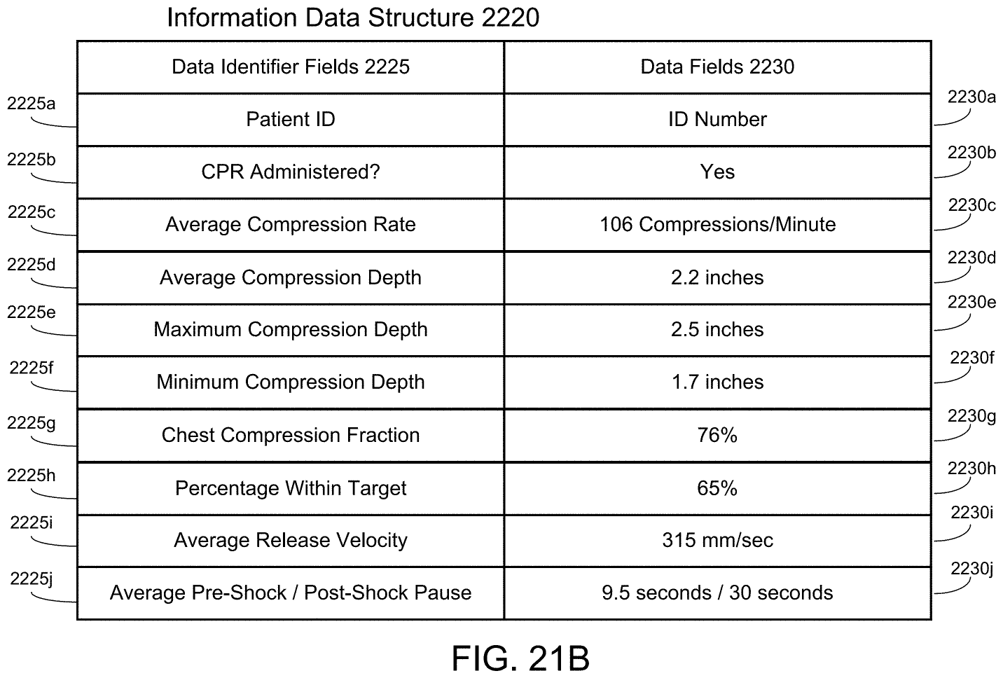

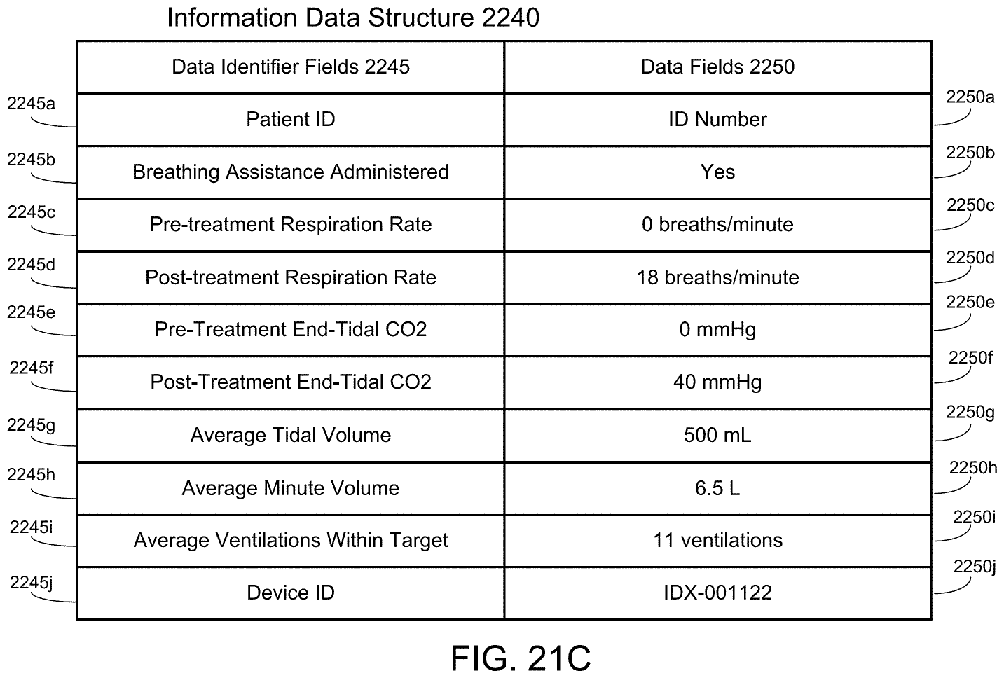

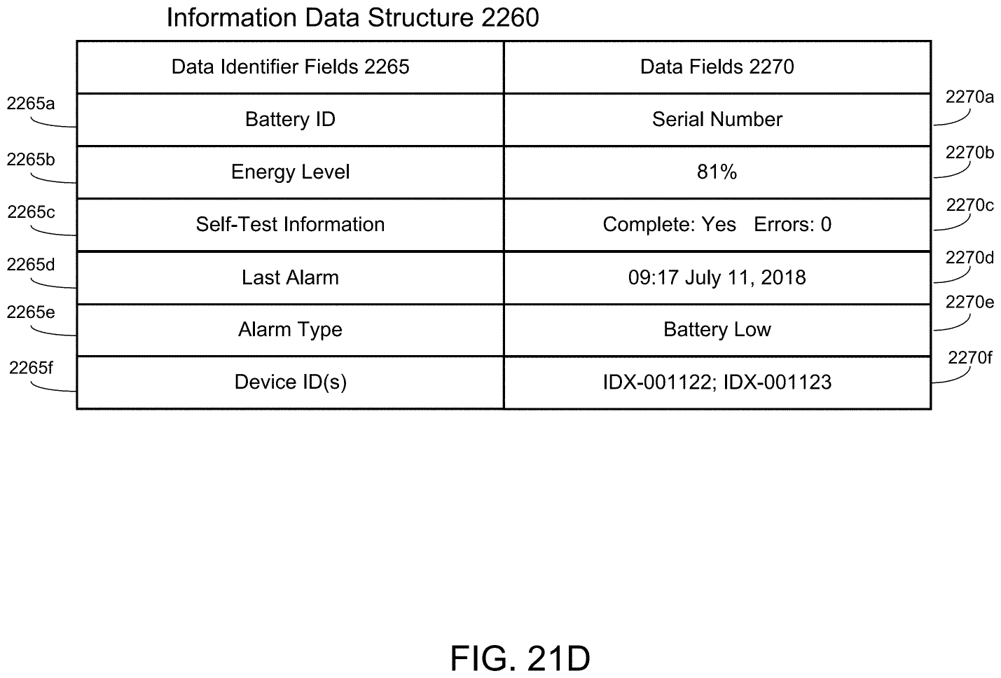

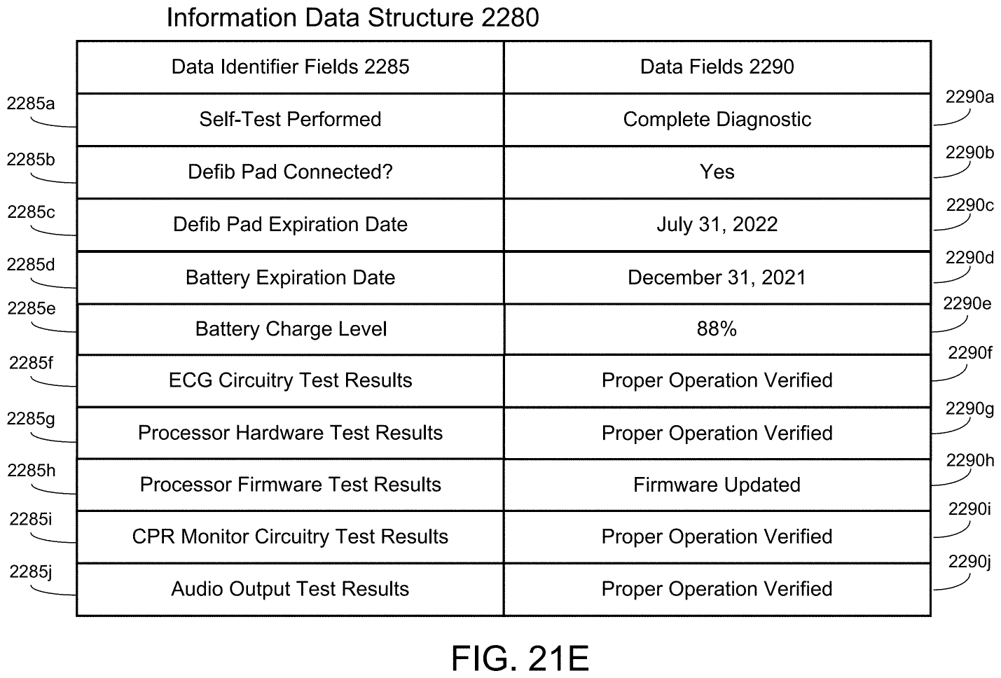

For example, FIGS. 21A-21E illustrate sample data structures that can be stored by a medical device such as those described herein. A portion of the information contained within the data structures can be transferred as a portion of a payload containing high-priority data as described herein. For example, as shown in FIG. 21A, data structure 2200 can include information related to monitoring and treatment of a patient using a defibrillator as described herein. As shown in FIG. 21A, the data structure 2200 can include various categories of fields such as data identifier fields 2205 and data fields 2210. Each of the categories of fields can have various fields nested therein. For example, data identifier fields 2205 can include a patient ID field 2205a, a pre-treatment heart rate field 2205b, a pre-treatment blood pressure field 2205c, a treatable arrhythmia field 2205d, a treatment shock provided field 2205e, a number of treatment shocks field 2205f, a first treatment shock information field 2205g, a second treatment shock information field 2205h, a post-treatment heart rate field 2205i, and a post-treatment blood pressure field 2205j. While data structures described herein may include static fields that provide particular values for certain categories, data structures may also include dynamic fields, where larger feeds of data such as physiological waveforms and other streams and/or snapshots of data are recorded over a continuous period of time. As further shown in FIG. 21A, each individual data identifier field 2205 can have a corresponding data field 2210 that includes additional information related to the identified piece of data. For example, ID number field 2210a can include a patient identification number such as the patient's social security number, patient record number, insurance information number, and/or other similar identification that can be used to identify the patient being treated. As shown in FIG. 21A, data field 2210b can include the patient's pre-treatment heart rate information, and data field 2210c can include the patient's pre-treatment blood pressure information. As further shown in FIG. 21A, data field 2210d can provide an indication of whether the patient's arrhythmia was treatable, data field 2210e can provide an indication as to whether at least one treatment shock was provided to the patient, and data field 2210f can provide an indication of how many treatment shocks were provided to the patient. As further shown in FIG. 21A, data field 2210g can provide information related to the first treatment shock such as energy delivered and whether the first treatment shock was successful, data field 2210h can provide information related to the second treatment shock such as energy delivered and whether the second treatment shock was successful, data field 2210i can provide an indication of the patient's post-treatment heart rate, and data field 2210j can provide an indication of the patient's post-treatment blood pressure.

Similarly, FIG. 21B illustrates a data structure related to monitoring and treatment of a patient using a chest compression monitoring device as described herein. As shown in FIG. 21B, the data structure 2220 can include various categories of fields such as data identifier fields 2225 and data fields 2230. Each of the categories of fields can have various fields nested therein. For example, data identifier fields 2225 can include a patient ID field 2225a, a CPR administered field 2225b, an average compression rate field 2225c, an average compression depth field 2225d, a maximum compression depth field 2225e, a minimum compression depth field 2225f, a chest compression fraction field 2225g, a percentage of chest compressions that are within a target depth field 2225h, an average release velocity field 2225i, and an average pre-shock/post-shock pause field 2225j. Or, in some embodiments, the data identifier fields and/or data structures may store the raw data associated with one or more of the parameters described herein, and the actual calculations for such parameters may be performed by a processor external to the accessory, such as a processor on a separate computing device, mobile device, and/or server.

As further shown in FIG. 21B, each individual data identifier field 2225 can have a corresponding data field 2230 that includes additional information related to the identified piece of data. For example, ID number field 2230a can include a patient identification number such as the patient's social security number, patient record number, insurance information number, and/or other similar identification that can be used to identify the patient being treated. As shown in FIG. 21B, data field 2230b can include an indication of whether CPR was administered to the patent, data field 2230c can include a calculated average compression rate, data field 2230d can include a calculated average compression depth, data field 2230e can include a measured maximum compression depth, data field 2230f can include a measured minimum compression depth, data field 2230g can include a calculated chest compression fraction, data field 2230h can include a percentage of chest compressions that were within a target range, data field 2230i can include an average release velocity measurement, and data field 2230j can include average pre-shock and post-shock pause measurements. In some examples, the data structure 2220 can include additional information such as device identification information including, for example, device serial number, device capability, and device type.

In another example, FIG. 21C illustrates a sample information data structure 2240 configured to store information collected from a ventilator including an airflow sensor as described herein. As shown in FIG. 21C, the data structure 2240 can include various categories of fields such as identifier fields 2245 and data fields 2250. Each of the categories of fields can have various fields nested herein. For example, data identifier fields 2245 can include a patient ID field 2245a, a breathing assistance administered field 2245b, a pre-treatment respiration rate field 2245c, a post-treatment respiration field 2245d, a pre-treatment end-tidal CO2 field 2245e, a post-treatment end-tidal CO2 field 2245f, average tidal volume 2245g, average minute volume 2245h, percentage of ventilations that are within a target range 2245i, and device ID 2245j.

As further shown in FIG. 21C, each individual data identifier field 2245 can have a corresponding data field 2250 that includes additional information related to the identified piece of data. For example, ID number field 2250a can include a patient identification number such as the patient's social security number, patient record number, insurance information number, and/or other similar identification that can be used to identify the patient being treated. As shown in FIG. 21C, data field 2250b can include an indication of whether breathing assistance was administered to the patent, data field 2250c can include a measured pre-treatment respiration rate, data field 2250d can include a measured post-treatment respiration rate, data field 2250e can include a measured pre-treatment end-tidal CO2 level, and data field 2250f can include a measured post-treatment end-tidal CO2 level. Data field 2250g can include a measured average tidal volume, data field 2250h can include an average minute volume, data field 2250i can include a percentage of ventilations that are within a target range, and data field 2250j can include device identification information.

FIG. 21D illustrates an information data structure 2260 configured to store device-specific information collected from a medical device as described herein. As shown in FIG. 21D, the data structure 2260 can include various categories of fields such as identifier fields 2265 and data fields 2270. Each of the categories of fields can have various fields nested herein. For example, data identifier fields 2265 can include a battery ID field 2265a, an energy level field 2265b, a self-test information field 2265c, a last alarm information field 2265d, an alarm type field 2265e, and a device ID field 2265f including an indication of what device or devices the battery has been previously used to power.

As further shown in FIG. 21D, each individual data identifier field 2265 can have a corresponding data field 2270 that includes additional information related to the identified piece of data. For example, battery serial number field 2270a can include a manufacturer assigned serial number for the battery. As shown in FIG. 21D, data field 2270b can include a current energy level for the battery, data field 2270c can include information related to the most recent self-test such as whether the test completed and if there were any errors, data field 2270d can include information related to the last alarm the battery issued, data field 2270e can include an indication of what type of alarm was issued, and data field 2270f can include one or more device ID numbers indicating what devices the battery has provided power to.

As noted above, data field 2270c can indicate whether a self-test has been performed by the medical device. The results of this self-test can also be included in an information data structure as described herein. For example, as shown in FIG. 21E, information data structure 2280 can be configured to store information collected during a self-test of a medical device. As shown in FIG. 21E, the data structure 2280 can include various categories of fields such as identifier fields 2285 and data fields 2290. Each of the categories of fields can have various fields nested herein. For example, data identifier fields 2285 can include a self-test performed field 2285a, a defibrillator pad connection field 2285b, a defibrillator pad expiration date field 2285c, a battery expiration date field 2285d, a battery charge level field 2285e, an ECG circuitry test results field 2285f, a processor hardware test results field 2285g, a processor firmware test results field 2285h, a CPR monitor circuitry test results field 2285i, and an audio output test results field 2285j.

As further shown in FIG. 21E, each individual data identifier field 2285 can have a corresponding data field 2290 that includes additional information related to the identified piece of data. For example, self-test performed field 2290a can include the name of the self-test performed by the medical device. As shown in FIG. 21E, data field 2290b can indicate whether the defibrillator pad was connected during the self-test, data field 2290c can indicate what the expiration date of the defibrillator pad is, data field 2290d can indicate what the expiration date of the battery is, data field 2290e can indicate the current battery charge level (represented, for example, as a percentage as shown in FIG. 21E), data field 2290f can indicate the ECG circuitry test results, data field 2290g can indicate the processor hardware test results, data field 2290h can indicate the processor firmware test results, data field 2290i can indicate the CPR monitor circuitry test results, and data field 2290j can indicate the audio output test results.

It should be noted that the individual data structures, data fields, and information contained in and illustrated by FIGS. 21A-E is provided by way of example only. It should also be noted that, in some examples above, the information contained in the data fields as shown in FIGS. 21A-21E includes calculated data. This is provided by way of example only. In some implementations, the medical device as described herein as collecting the information may not have the processing capabilities to calculate specific information such as that shown in FIGS. 21A-21E and described above. In such implementations, the medical device can provide raw recorded data to another processing device as described herein for calculation of the actual values as described herein.

Referring again to FIG. 17, the processor of the medical device can encrypt 1704 the payload including the high priority data to generate encrypted data. For example, the payload can include a portion of the data as shown in FIGS. 21A-21E and described above. The processor of the medical device can render 1706 the encrypted data via one or more QR codes as described herein.

As further shown in FIG. 17, a processor of the mobile computing device can acquire 1708 one or more images of the QR codes as displayed by the medical device. The processor of the mobile computing device can convert 1710 the QR codes to encrypted data by decoding the information contained within the QR code. The processor of the mobile computing device can decrypt 1712 the encrypted data using, for example, a decryption key to generate decrypted data. The processor of the mobile computing device can further process 1714 the decrypted data to access the high priority data as identified by the processor of the medical device. The processor of the mobile computing device can use the high priority data to establish 1716 a network connection with the medical device as described above, for example, in the discussion of FIG. 16. Additionally, the processor of the mobile computing device can use the high priority data to display 1718 patient information such as CPR performance metrics, defibrillator treatment information, ventilator information, and other patient information included in the payload from the medical device.

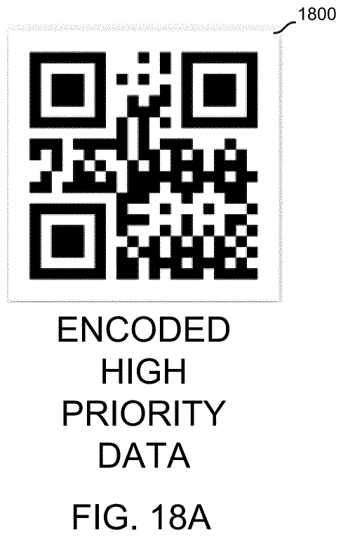

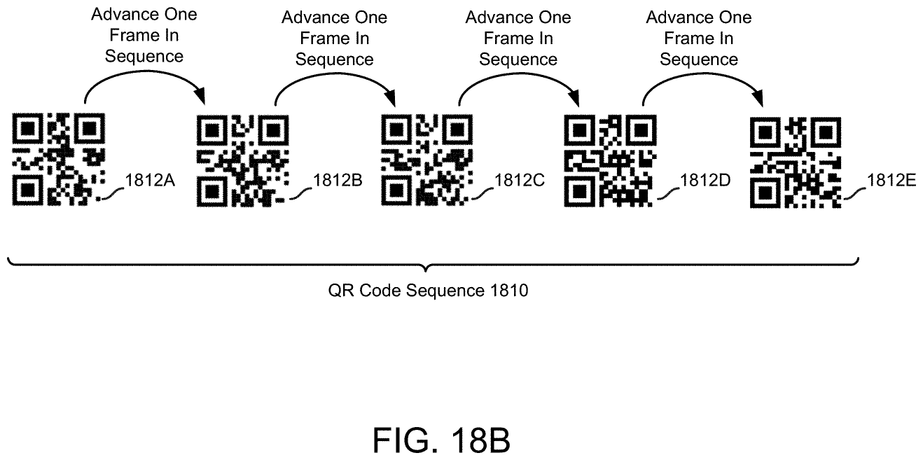

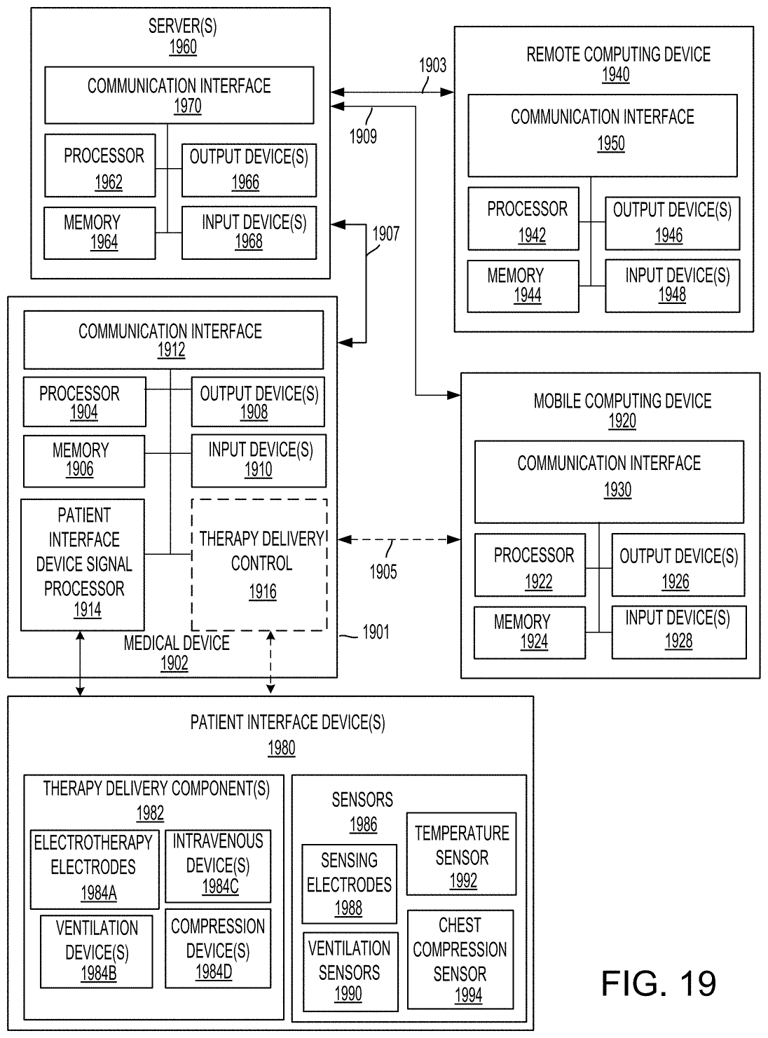

As described herein, QR codes can be used to represent various information such as high priority data, static data, dynamic data, and other similar data as described herein. Based upon the configuration of the QR code, a particular QR code may be configured to include high priority data as described above in the discussion of FIG. 17. For example, as shown in FIG. 18A, a QR code 1800 can be specifically encoded to include high priority data such as patient information, network connectivity information, patient demographic information, and other similar sensitive information that may be determined to include high priority data. In certain implementations, the general appearance of a high priority data QR code may be similar or identical to a low priority information QR code. However upon decoding the QR code by, for example, a processor of a mobile computing device can identify encrypted data within the QR code that is related to high priority data.

Additionally, as described herein, for example, in the discussion of FIG. 2A above, a series or sequence of QR codes may be displayed buy a medical device as described herein. For example, as shown in FIG. 18B, a QR code sequence 1810 can be displayed over a period of time such that an individual QR code displayed by a medical device updates over time. As such, the medical device can display or otherwise output a series and/or sequence of QR codes via a display on the medical device. As shown in FIG. 18B, an initial QR code 1812A can be displayed by the medical device. After a period of time, the QR code sequence 1810 can advance one frame, and the medical device display can now output a QR code 1812B. Similarly, after a period of time, the QR code sequence 1810 can advance another frame, and the medical device display can now output a QR code 1812C. As further shown in FIG. 18B, the QR code sequence 1810 can also include a QR code 1812D and a QR code 1812E. In certain implementations, the medical device can be configured to repeat the sequence of QR codes. In other examples, the individual QR codes contained within the sequence can be updated such that continually updated information is provided in the QR code sequences.

It should be noted that five individual QR codes is shown in the QR code sequence 1810 as shown in FIG. 18B is provided by way of example only. Depending upon the amount of information to convey to a mobile computing device via the QR codes, the medical device can generate a QR code sequence having a length determined based upon the amount of information to include in the QR codes. For example, if each QR code is configured to store 1,000 bytes of information, and the medical device has 24,000 bytes of information to transmit, the processor of the medical device can generate a sequence of 24 unique QR codes to output to the mobile computing device, thereby transmitting all 24,000 bytes in a single QR code sequence.

Each of the processes disclosed herein depicts one particular sequence of operations in a particular example. Some operations are optional and, as such, can be omitted in accord with one or more examples. Additionally, the order of operations can be altered, or other operations can be added, without departing from the scope of the apparatus and methods discussed herein. It should be noted that, in some examples, one or more of the processes disclosed herein are stored as sequences of instructions that are executable by a processor. In these examples, the sequences of instructions can be stored in one or more non-volatile/non-transitory data storage media accessible by the processor. The processor and/or data storage media can be a part of a medical device, as described herein.

Referring now to FIG. 19, a block diagram of examples of computing and medical device components are shown schematically.

The medical device 1902 can include a processor 1904, a memory 1906, one or more output devices 1908, one or more user input devices 1910, and a communications interface 1912. The communications interface 1912 can include any of a variety of transmitters and/or receivers. For instance, in some examples, the communications interface 1912 includes one or more of an NFC tag, an RFID tag, a barcode, and a QR code.

In various implementations, the medical device 1902 can be a defibrillator, patient monitor, defibrillator/monitor, an automated compression device, a therapeutic cooling device, an extracorporeal membrane oxygenation (ECMO) device, a ventilation device, combinations thereof, or another type of medical device configured to couple to one or more therapy delivery components to provide therapy to the patient. In an implementation, the medical device 1902 can be an integrated therapy delivery/monitoring device within a single housing 1901. The single housing 1901 can surround, at least in part, a patient interface device signal processor 1914 and/or a therapy delivery control module 1916.

The patient interface device(s) 1980 can include one or more therapy delivery component(s) 1982 and/or one or more sensor device(s) 1986. The medical device 1902 can be configured to couple to the one or more therapy delivery component(s) 1982. In combination, the medical device 1902 and the one or more therapy delivery components can provide therapeutic treatment to a patient. In an implementation, the medical device 1902 can include or incorporate the therapy delivery component(s) 1982. The therapy delivery component(s) 1982 are configured to deliver therapy to the patient and can be configured to couple to the patient. For example, the therapy delivery component(s) 1982 can include one or more of electrotherapy electrodes including defibrillation electrodes and/or pacing electrodes, chest compression devices (e.g., one or more belts or a piston), ventilation devices (e.g., a mask and/or tubes), drug delivery devices, etc. The medical device 1902 can include the one or more therapy delivery component(s) 1982 and/or can be configured to couple to the one or more therapy delivery component(s) 1982 in order to provide medical therapy to the patient. The therapy delivery component(s) 1982 can be configured to couple to the patient. For example, a health care provider may attach the electrodes to the patient, and the medical device 1902 (e.g., a defibrillator or defibrillator/patient monitor) may provide electrotherapy to the patient via the defibrillation electrodes. These examples are not limiting of the disclosure as other types of medical devices, therapy delivery components, sensors, and therapy are within the scope of the disclosure.

The medical device 1902 can be, for example, a therapeutic medical device capable of delivering a medical therapy. For example, the medical therapy can be electrical therapy (e.g. defibrillation, cardiac pacing, synchronized cardioversion, diaphragmatic or phrenic nerve stimulation) and the medical device 1902 can be a defibrillator, a defibrillator/monitor and/or another medical device configured to provide electrotherapy. As another example, the medical therapy can be chest compression therapy for treatment of cardiac arrest and the first medical device 1902 can be a mechanical chest compression device such as a belt-based chest compression device or a piston-based chest compression device. As other examples, the medical therapy can be ventilation therapy, therapeutic cooling or other temperature management, invasive hemodynamic support therapy (e.g. Extracorporeal Membrane Oxygenation (ECMO)), etc. and the medical device 1902 can be a device configured to provide a respective therapy. In an implementation, the medical device 1902 can be a combination of one or more of these examples. The therapeutic medical device can include patient monitoring capabilities via one or more sensors. These types of medical therapy and devices are examples only and not limiting of the disclosure.

The medical device 1902 can include, incorporate, and/or be configured to couple to the one or more sensor(s) 1986 which can be configured to couple to the patient. The sensor(s) 1986 are configured to provide signals indicative of sensor data to the medical device 1902. The sensor(s) 1986 can be configured to couple to the patient. For example, the sensor(s) 1986 can include cardiac sensing electrodes, a chest compression sensor, and/or ventilation sensors. The one or more sensors 1986 can generate signals indicative of physiological parameters of the patient. For example, the physiological parameters can include one or more of at least one vital sign, an ECG, blood pressure, heart rate, pulse oxygen level, respiration rate, heart sounds, lung sounds, respiration sounds, tidal CO2, saturation of muscle oxygen (SMO2), arterial oxygen saturation (SpO2), cerebral blood flow, electroencephalogram (EEG) signals, brain oxygen level, tissue pH, tissue fluid levels, physical parameters as determined via ultrasound images, parameters determined via near-infrared reflectance spectroscopy, pneumography, and/or cardiography, etc. Additionally or alternatively, the one or more sensors 1986 can generate signals indicative of chest compression parameters, ventilation parameters, drug delivery parameters, fluid delivery parameters, etc.

In addition to delivering therapy to the patient, the therapy delivery component(s) 1982 can include, be coupled to, and/or function as sensors and provide signals indicative of sensor data (e.g., second sensor data) to the medical device 1902. For example, the defibrillation electrodes can be configured as cardiac sensing electrodes as well as electrotherapy delivery devices and can provide signals indicative of transthoracic impedance, ECG, heart rate and/or other physiological parameters. As another example, a therapeutic cooling device can be an intravenous cooling device. Such a cooling device can include an intravenous (IV) device as a therapy delivery component configured to deliver cooling therapy and sense the patient's temperature. For example, the IV device can be a catheter that includes saline balloons configured to adjust the patient's temperature via circulation of temperature controlled saline solution. In addition, the catheter can include a temperature probe configured to sense the patient's temperature. As a further example, an IV device can provide therapy via drug delivery and/or fluid management. The IV device can also monitor and/or enabling monitoring of a patient via blood sampling and/or venous pressure monitoring (e.g., central venous pressure (CVP) monitoring).

The medical device 1902 can be configured to receive the sensor signals (e.g., from the therapy delivery component(s) 1982 and/or the sensor(s) 1986) and to process the sensor signals to determine and collect the patient data. The patient data can include patient data which can characterize a status and/or condition of the patient (e.g., physiological data such as ECG, heart rate, respiration rate, temperature, pulse oximetry, non-invasive hemoglobin parameters, capnography, oxygen saturation (SpO2), end tidal carbon dioxide (EtCO2), invasive blood pressure (IBP), non-invasive blood pressures (NIBP), tissue pH, tissue oxygenation, Near Infrared Spectroscopy (NIRS) measurements, etc.). Additionally or alternatively, the patient data can characterize the delivery of therapy (e.g., chest compression data such as compression depth, compression rate, etc.) and/or the patient data can characterize a status and/or condition of the medical equipment used to treat the patient (e.g., device data such as shock time, shock duration, attachment of electrodes, power-on, etc.).

The components of 1904, 1906, 1908, 1910, 1912, and 1916 of the medical device 1902 are communicatively coupled (directly and/or indirectly) to each other for bi-directional communication.

Although shown as separate entities in FIG. 19, the one or more of the components of the medical device 1902 can be combined into one or more discrete components and/or can be part of the processor 1904. The processor 1904 and the memory 1906 can include and/or be coupled to associated circuitry to perform the functions described herein.

In an implementation, the medical device 1902 can be a therapeutic medical device configured to deliver medical therapy to the patient. Thus, the medical device 1902 can optionally include the therapy delivery control module 1916. For example, the therapy delivery control module 1916 can be an electrotherapy delivery circuit that includes one or more capacitors configured to store electrical energy for a pacing pulse or a defibrillating pulse. The electrotherapy delivery circuit can further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 1916 can be a compression device electro-mechanical controller configured to control a mechanical compression device. As a further example, the therapy delivery control module 1916 can be an electro-mechanical controller configured to control drug delivery, temperature management, ventilation, and/or other type of therapy delivery. Alternatively, some examples of the medical device 1902 may not be configured to deliver medical therapy to the patient but can be configured to provide patient monitoring and/or diagnostic care. As shown in FIG. 19, in some examples, the therapy delivery control module 1916 exchanges messages 1905 with the mobile computing device 1920 (e.g., the patient mobile computing device). These messages can include patient data descriptive of therapy provided to the patient or other patient data stored on the medical device 1902.

The medical device 1902 can incorporate and/or be configured to couple to one or more patient interface device(s) 1980. The patient interface device(s) 1980 can include one or more therapy delivery component(s) 1982 and one or more sensor(s) 1986. The one or more therapy delivery component(s) 1982 and the one or more sensor(s) 1986 sensor can provide one or more signals to the medical device 1902 via wired and/or wireless connection (s).

The one or more therapy delivery component(s) 1982 can include electrotherapy electrodes (e.g., the electrotherapy electrodes 1984a), ventilation device(s) (e.g., the ventilation devices 1984b), intravenous device(s) (e.g., the intravenous devices 1984c), compression device(s) (e.g., the compression devices 1984d), etc. For example, the electrotherapy electrodes can include defibrillation electrodes, pacing electrodes, and/or combinations thereof. The ventilation devices can include a tube, a mask, an abdominal and/or chest compressor (e.g., a belt, a cuirass, etc.), etc. and combinations thereof. The intravenous devices can include drug delivery devices, fluid delivery devices, and combinations thereof. The compression devices can include mechanical compression devices such as abdominal compressors, chest compressors, belts, pistons, and combinations thereof. In various implementation, the therapy delivery component(s) 1982 can be configured to provide sensor data and/or be coupled to and/or incorporate sensors. For example, the electrotherapy electrodes can provide sensor data such as transthoracic impedance, ECG, heart rate, etc. Further the electrotherapy electrodes can include and/or be coupled to a chest compression sensor. As another example, the ventilation devices can be coupled to and/or incorporate flow sensors, gas species sensors (e.g., oxygen sensor, carbon dioxide sensor, etc.), etc. As a further example, the intravenous devices can be coupled to and/or incorporate temperature sensors, flow sensors, blood pressure sensors, etc. As yet another example, the compression devices can be coupled to and/or incorporate chest compression sensors, patient position sensors, etc. The therapy delivery control module 1916 can be configured to couple to and control the therapy delivery component(s) 1982.

In various implementations, the sensor(s) 1986 can include one or more sensor devices configured to provide sensor data that includes, for example, but not limited to ECG, blood pressure, heart rate, pulse oxygen level, respiration rate, heart sounds, lung sounds, respiration sounds, tidal CO2, saturation of muscle oxygen (SMO2), arterial oxygen saturation (SpO2), cerebral blood flow, electroencephalogram (EEG) signals, brain oxygen level, tissue pH, tissue fluid levels, images and/or videos via ultrasound, laryngoscopy, and/or other medical imaging techniques, near-infrared reflectance spectroscopy, pneumography, cardiography, and/or patient movement. Images and/or videos can be two-dimensional or three-dimensional.

The sensor(s) 1986 can include sensing electrodes (e.g., the sensing electrodes 1988), ventilation sensors (e.g., the ventilation sensors 1990), temperature sensors (e.g., the temperature sensor 1992), chest compression sensors (e.g., the chest compression sensor 1994), etc. For example, the sensing electrodes can include cardiac sensing electrodes. The cardiac sensing electrodes can be conductive and/or capacitive electrodes configured to measure changes in a patient's electrophysiology, for example to measure the patient's ECG information. In an implementation, the sensing electrodes can be configured to measure the transthoracic impedance and/or a heart rate of the patient. The ventilation sensors can include spirometry sensors, flow sensors, pressure sensors, oxygen and/or carbon dioxide sensors such as, for example, one or more of pulse oximetry sensors, oxygenation sensors (e.g., muscle oxygenation/pH), O₂ gas sensors and capnography sensors, and combinations thereof. The temperature sensors can include an infrared thermometer, a contact thermometer, a remote thermometer, a liquid crystal thermometer, a thermocouple, a thermistor, etc. and can measure patient temperature internally and/or externally. The chest compression sensor can include one or more motion sensors including, for example, one or more accelerometers, one or more force sensors, one or more magnetic sensors, one or more velocity sensors, one or more displacement sensors, etc. The chest compression sensor can be, for example, but not limited to, a compression puck, a smart phone, a hand-held device, a wearable device, etc. The chest compression sensor can be configured to detect chest motion imparted by a health care provider and/or an automated chest compression device (e.g., a belt system, a piston system, etc.). The chest compression sensor can provide signals indicative of chest compression data including displacement data, velocity data, release velocity data, acceleration data, compression rate data, dwell time data, hold time data, blood flow data, blood pressure data, etc. In an implementation, the sensing electrodes and/or the electrotherapy electrodes can include or be configured to couple to the chest compression sensor.

Continuing with FIG. 19, examples of components of the mobile computing device 1920 are shown schematically. In an implementation, the mobile computing device 1920 can be configured as a mobile computing device. The mobile computing device 1920 can include a processor 1922, a memory 1924, one or more output devices 1926, one or more user input devices 1928, and a communications interface 1930. FIG. 19 also illustrates schematically examples of components of the remote computing device 1940. As shown in FIG. 19, remote the computing device 1940 can include a processor 1942, a memory 1944, one or more output devices 1946, one or more user input devices 1948, and a communications interface 1950. FIG. 19 further illustrates schematically examples of components of the server(s) 1960. As shown in FIG. 19, the server(s) 1960 can include a processor 1962, a memory 1964, one or more output devices 1966, one or more user input devices 1968, and a communications interface 1970.

Each of the mobile computing device 1920 (e.g., the mobile computing device) and the remote computing device 1940 can be a computer system, such as a desktop, notebook, mobile, portable, or other type of computing system. Each of these devices 1920 and 1940 can include server(s) and/or access server(s) via a monitor and/or other connected user interface device. Although described as server(s), the server(s) 1960 can be another type of computing system including for example a desktop, notebook, mobile, portable, or other type of computing system.

As shown in FIG. 19, each of the devices 1920 and 1940, along with the server(s) 1960 and the medical device 1902, includes a bus or other interconnection mechanism that communicably couples the processor, memory, output devices, input devices, and communication interface included therein. The bus can include a PCI/PCI-X or SCSI based system bus depending on the storage devices used, for example.

The processors 1904, 1922, 1942, and 1962 can each include a processor, such as, but not limited to, an Intel® Itanium® or Itanium 2® processor(s), or AMD® Opteron® or Athlon MP® processor(s), or Motorola® lines of processors. The communication interfaces 1912, 1950, 1930, and 1970 can each be any of an RS-232 port for use with a modem-based dialup connection, a 10/100 Ethernet port, or a Gigabit port using copper or fiber, for example. The communication interfaces 1912, 1950, 1930, and 1970 may be chosen depending on a network(s) such a Local Area Network (LAN), Wide Area Network (WAN), or any network to which the medical device 1902, the mobile computing device 1920, the remote computing device 1940, and/or the server(s) 1960 may connect. The memories 1906, 1944, 1924, and 1964 can be Random Access Memory (RAM), Read Only Memory (ROM), Flash memory, and/or another dynamic volatile and/or non-volatile storage device(s). The memories 1906, 1944, 1924, and 1964 can be used to store information and instructions. For example, hard disks such as the Adaptec® family of SCSI drives, an optical disc, an array of disks such as RAID (e.g. the Adaptec family of RAID drives), or any other mass storage devices may be used. The components described above are meant to exemplify some types of possibilities. In no way should the aforementioned examples limit the scope of the disclosure. The memories 1906, 1944, 1924, and 1964 can further include removable storage media such as external hard-drives, floppy drives, flash drives, IOMEGA® Zip Drives, Compact Disc—Read Only Memory (CD-ROM), Compact Disc—Re-Writable (CD-RW), or Digital Video Disk—Read Only Memory (DVD-ROM), for example.

Continuing with FIG. 19, the server(s) 1960 can include, for example, the one or more storage server(s) and one or more application server(s). In some examples, the server(s) 1960 are configured to exchange messages 1903 with the remote computing device 1940. These messages can include sensor and/or treatment data as described above. In some examples, the server(s) 1960 are configured to exchange messages 1907 with the medical device 1902. These messages can include data descriptive of a patient being treated via by the medical device and/or treatment being delivered by the medical device 1902.

Some examples of the present disclosure include various steps, some of which can be performed by hardware components or can be embodied in machine-executable instructions. These machine-executable instructions can be stored on a non-transitory data storage medium and can be used to cause a general-purpose or a special-purpose processor programmed with the instructions to perform the steps. The non-transitory data storage medium can further to store an operating system and the machine-executable instructions can be included within one or more software applications or programs. These programs can implement the features disclosed herein and the methods that they execute. Alternatively, the steps can be performed by a combination of hardware, software, and/or firmware, on one device and/or distributed across multiple devices and/or processors. In addition, some examples of the present disclosure can be performed or implemented, at least in part (e.g., one or more modules), on one or more computer systems, mainframes (e.g., IBM mainframes such as the IBM zSeries, Unisys ClearPath Mainframes, HP Integrity NonStop server(s), NEC Express series, and others), or client-server type systems. In addition, specific hardware aspects of examples of the present disclosure can incorporate one or more of these systems, or portions thereof.

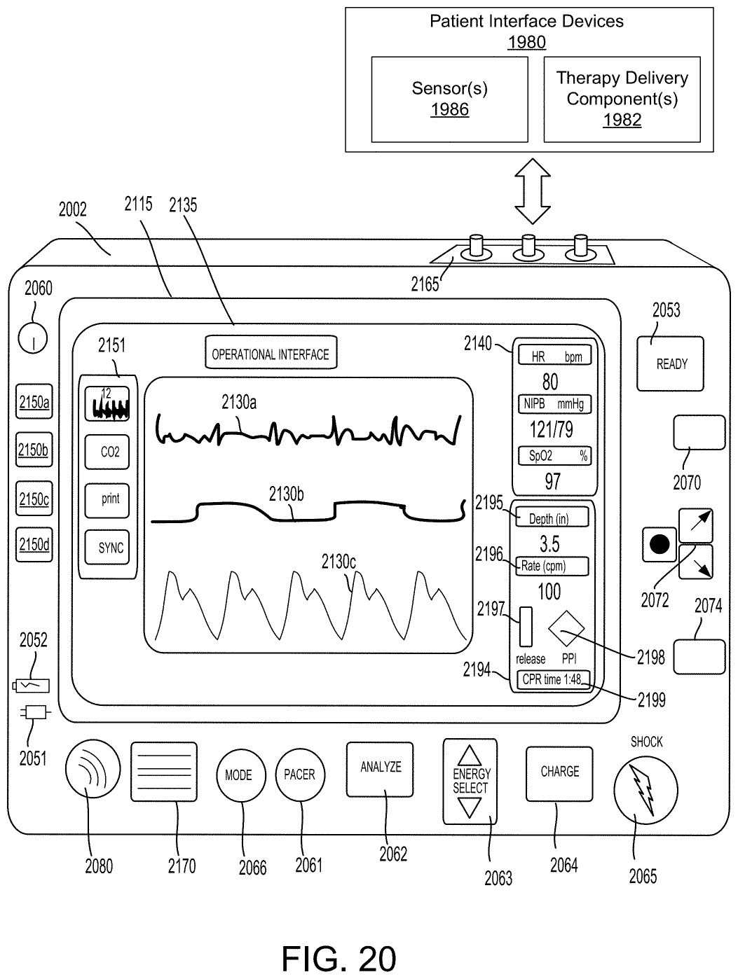

Referring to FIG. 20, an example of the medical device 1902 of FIG. 19 with an operational interface is shown. This example, the medical device 2002, is a patient monitor/defibrillator. This configuration of a medical device is an example only and not limiting of the disclosure. In various implementations, the medical device 2002 may be a defibrillator, patient monitor, defibrillator/monitor, an automated compression device, a therapeutic cooling device, an extracorporeal membrane oxygenation (ECMO) device, a ventilation device, combinations thereof, or another type of medical device configured to couple to one or more therapy delivery components to provide therapy to the patient. In an implementation, the medical device 2002 may be an integrated therapy delivery/monitoring device that includes a single housing. The single housing may surround, at least in part, the therapy delivery components and the monitoring components. In an implementation, the medical device 2002 may be a modular therapy delivery/monitoring device.

The medical device 2002 may include one or more output or input/output devices, for example, a display screen 2115. A processor of the medical device 2002 may control the display screen 2115 to selectively display the operational interface 2135. The operational interface 2135 as shown in FIG. 20 is an example only and elements may be rearranged, combined, altered, or deleted. As discussed in further detail below, selective display refers to the ability of the processor to select amongst various available display modes which may include an operational interface only display mode.

The operational interface 2135 may provide patient data received by the medical device 2002 from the patient interface device(s) 1980 (e.g., the therapy delivery component(s) 1982 and/or from the sensor(s) 1986). For example, the medical device 2002 may be configured to couple to the patient interface device(s) 1980 via the one or more connection ports 2165. The operational interface 2135 may provide the patient data in real-time as the signals are received and processed by the processor 1904 of the medical device 2002.

The therapy delivery component(s) 1982 are configured to deliver therapy to the patient and may be configured to couple to the patient. For example, the therapy delivery component(s) 1982 may include one or more of electrotherapy electrodes including defibrillation electrodes and/or pacing electrodes, chest compression devices, ventilation devices, drug delivery devices, etc. In addition to delivering therapy to the patient, the therapy delivery component(s) 1982 may include, be coupled to, and/or function as sensors and provide signals indicative of sensor data (e.g., first sensor data) to the medical device 2002. For example, the therapy delivery component(s) 1982 may be defibrillation and/or pacing electrodes and may provide signals indicative of transthoracic impedance, ECG, heart rate and/or other physiological parameters.

The sensor(s) 1986 are configured to provide signals indicative of sensor data (e.g., second sensor data) to the medical device 2002. The sensor(s) 1986 may be configured to couple to the patient. For example, the sensor(s) 1986 may include cardiac sensing electrodes, a chest compression sensor, and/or ventilation sensors.

The medical device 2002 may be configured to receive the sensor signals (e.g., from the therapy delivery component(s) 1982 and/or the sensor(s) 1986) indicative of patient data for the patient and configured to process the sensor signals to determine and collect the patient data. The patient data may include patient data which may characterize a status and/or condition of the patient (e.g., physiological data such as ECG, heart rate, pulse oximetry, non-invasive hemoglobin parameters, capnography, oxygen and CO2 concentrations in the airway, invasive and non-invasive blood pressures, tissue pH, tissue oxygenation, near infra-red spectroscopy, etc.). Additionally or alternatively, the patient data may characterize the delivery of therapy (e.g., chest compression data such as compression depth, compression rate, etc.) and/or the patient data may characterize a status and/or condition of the medical equipment used to treat the patient (e.g., device data such as shock time, shock duration, attachment of electrodes, power-on, etc.).

In addition to the display screen 2115, the medical device 2002 may include one or more other output devices such as, for example, a speaker 2170. The processor 1904 may be configured to control the speaker 2170 to provide audible instructions, a metronome (e.g., a chest compression metronome), feedback, and/or physiological information for a user of the medical device 2002. The medical device 2002 may further include device status indicators and/or device operation controls. For example, device status indicators may include a power-on indicator 2051, a battery charge indicator 2052, and/or a device ready indicator 2053. The device operation controls may include a power-on control 2060, a pacer mode control 2061, a heart rhythm analyze control 2062, a defibrillation energy selection control 2063, a charge control 2064, a shock delivery control 2065, an alarm control 2070, one or more display navigation controls 2072, and a sensor control 2074. Activation of the sensor control 2074 may cause an associated patient data sensor to capture patient data and provide the data to the medical device 2002. The display screen 2115 may provide the captured patient data. For example, activation of the sensor control 2074 may cause a blood pressure sensor to measure the patient's blood pressure and may cause the operational interface 2135 to display the measured blood pressure in response to activation of the sensor control 2074. The medical device 2002 may include one or more soft-keys 2150a, 2150b, 2150c, 2150d, one or more soft-key labels 2151, and/or an NFC tag 2080. The NFC tag 2080 may enable the medical device 2002 to communicatively couple with another device, such as the mobile computing device 1920.

Having thus described several aspects of at least one example, it is to be appreciated that various alterations, modifications, and improvements will readily occur to those skilled in the art. For instance, examples disclosed herein can also be used in other contexts. Such alterations, modifications, and improvements are intended to be part of this disclosure and are intended to be within the scope of the examples discussed herein. Accordingly, the foregoing description and drawings are by way of example only.

1. A medical system comprising:
a medical device comprising
at least one physiologic sensor configured to acquire physiological signals from a patient,
at least one processor coupled to the at least one physiologic sensor, and
at least one optical code encoded with encrypted data; and
a mobile computing device comprising
a camera, and
one or more processors coupled to the camera and configured to
acquire one or more images of the at least one optical code,
decode the one or more images of the at least one optical code to generate a copy of the encrypted data,
decrypt the copy of the encrypted data to generate decrypted data, and
process the decrypted data to establish an operable connection between the mobile computing device and the medical device.

2. The medical system of claim 1, wherein
the medical device further comprises at least one network interface coupled to the at least one processor,
the mobile computing device comprises one or more network interfaces coupled to the one or more processors,
the encrypted data comprises network connection information, and
to process the decrypted data comprises to establish a connection between the mobile computing device and the medical device via the one or more network interfaces and the at least one network interface using the network connection information.

3. The medical system of claim 2, wherein the network connection information comprises one or more of security credentials, an identifier of the medical device, or an identifier of a network associated with the medical device.

4. The medical system of claim 2, wherein the medical device is further configured to exchange information with the mobile computing device, the exchanged information comprising at least one of device readiness information, caregiver performance data, physiological data, or event marker data.

5. The medical system of claim 1, wherein the medical device comprises one or more of an automated external defibrillator, a defibrillator/monitor, a wearable defibrillator, a ventilator, a resuscitation system, a cardiac monitoring device, or a cardiopulmonary resuscitation (CPR) monitoring device.

6. The medical system of claim 1, wherein the medical device further comprises at least one display coupled to the at least one processor and the at least one processor is configured to:
encrypt sensitive data to generate the encrypted data;
encode the encrypted data and public data within the at least one optical code; and
output, via the at least one display, the at least one optical code encoded with the encrypted data and the public data.

7. The medical system of claim 6, wherein to decode the one or more images comprises to decode the one or more images of the at least one optical code to generate the copy of the encrypted data and a copy of the public data, and the one or more processors are further configured to process the copy of the public data.

8. The medical system of claim 1, wherein the medical device further comprises at least one display coupled to the at least one processor and the at least one processor is configured to:
encrypt data to generate the encrypted data; and
output, via the at least one display, the at least one optical code encoded with the encrypted data.

9. The medical system of claim 8, wherein
the medical device further comprises at least one network interface coupled to the at least one processor,
the mobile computing device comprises one or more network interfaces coupled to the one or more processors,
the data comprises network connection information, and
to process the decrypted data comprises to establish a connection between the mobile computing device and the medical device via the one or more network interfaces and the at least one network interface using the network connection information.

10. The medical system of claim 8, wherein
the mobile computing device further comprises a user interface,
the data comprises encounter information, and
to process the decrypted data comprises to output the encounter information via the user interface.

11. The medical system of claim 10, wherein the at least one physiologic sensor comprises at least one electrocardiogram (ECG) sensor configured to acquire transcutaneous ECG signals from the patient and the encounter information specifies one or more of initiation time and duration of an encounter documented by the encounter information, an arrhythmia condition of the patient detected during the encounter, treatment administered to the patient during the encounter, or efficacy of the treatment administered to the patient during the encounter.

12. The medical system of claim 11, wherein the medical device further comprises at least one therapy electrode configured to discharge transcutaneous electrotherapy to a myocardium of the patient and the encounter information further specifies one or more of a number of discharges administered to the patient during the encounter and whether the any of the one or more discharges resulted in conversion of the arrhythmia condition of the patient.

13. The medical system of claim 10, wherein the medical device further comprises at least one treatment sensor configured to monitor delivery of CPR to the patient and the encounter information specifies one or more of initiation time and duration of an encounter documented by the encounter information, emergency medical responder name information, CPR performance of the emergency medical responder, compression data and averages for a period of time, target treatment information, post shock pause values, pre-shock pause values, total length of treatment provided, or efficacy of treatment administered to the patient during the encounter.

14. The medical system of claim 10, wherein the medical device further comprises a ventilator including at least one flow sensor and at least one pressure sensor configured to measure air flow rates delivered to the patient and the encounter information specifies one or more of initiation time and duration of an encounter documented by the encounter information, breaths per minute as delivered to the patient and measured by the at least one flow sensor, air volume per breath as delivered to the patient and measured by the at least one flow sensor, air volume exhausted by the patient as measured by the at least one flow sensor, ventilator settings, or efficacy of treatment administered to the patient during the encounter.

15. The medical system of claim 8, wherein
the mobile computing device further comprises a user interface,
the data comprises device readiness information, and
to process the decrypted data comprises to output the device readiness information via the user interface.

16. The medical system of claim 15, wherein the device readiness information specifies one or more of a result of a self-test executed by the medical device, electrode expiration information, an amount of power remaining in a battery of the medical device, or status of network connectivity of the medical device.

17. The medical system of claim 8, wherein to output the at least one optical code comprises to encode the encrypted data within the at least one optical code.

18. The medical system of claim 17, wherein the at least one optical code comprises a plurality of optical codes.

19. The medical system of claim 1, wherein
the mobile computing device further comprises one or more light emitting devices coupled to the one or more processors,
the medical device further comprises at least one light sensor,
the one or more processors are further configured to
encode new data as one or more modulations of the one or more light emitting devices, and
transmit the one or more modulations, and
the at least one processor is configured to
acquire the one or more modulations via the at least one light sensor, and
demodulate the one or more modulations to generate a copy of the new data for processing.

20. The medical system of claim 1, wherein the medical device further comprises at least one light emitting device positioned within the at least one optical code and the at least one processor is configured to:
encrypt additional data to generate additional encrypted data;
encode the additional encrypted data as a plurality of modulations of the at least one light emitting device; and
transmit, via the at least one light emitting device, the plurality of modulations.

21. The medical system of claim 20, wherein
the medical device further comprises at least one network interface coupled to the at least one processor,
the mobile computing device further comprises
a user interface coupled to the one or more processors, and
one or more network interfaces coupled to the one or more processors,
the data comprises network connection information,
the additional data comprises encounter information,
to process the decrypted data comprises to establish a connection between the mobile computing device and the medical device via the one or more network interfaces and the at least one network interface using the network connection information, and
the one or more processors are further configured to
demodulate the plurality of modulations to generate a copy of the additional encrypted data,
decrypt the copy of the additional encrypted data to generate a copy of the additional data, and
output, via the user interface, a copy of the encounter information from the copy of the additional data.

22. The medical system of claim 1, wherein the mobile computing device further comprises one or more network interfaces coupled to the one or more processors and the one or more processors are further configured to:
receive a request for the encrypted data from a trusted mobile computing device via the one or more network interfaces; and
transmit, in response to reception of the request, the encrypted data to the trusted mobile computing device via the one or more network interfaces.

23. The medical system of claim 22, wherein the trusted mobile computing device is a wearable device.

24. The medical system of claim 1, wherein the mobile computing device further comprises a user interface and the one or more processors are further configured to:
receive a request for the at least one optical code from a trusted mobile computing device; and
display, in response to reception of the request, the at least one optical code via the user interface.

25.-56. (canceled)
